(19) United States
(12) Patent Application Publication
(10) Pub. No.: US 2026/0049078 A1
(43) Pub. Date: Feb. 19, 2026

(54) DIFLUORO- AND TRIFLUORO-ACETYL HYDRAZIDES AS SELECTIVE HDAC6 INHIBITORS

(71) Applicant: ITALFARMACO S.P.A., Milano (IT)

(72) Inventors: Mattia MARCHINI, Milano, MI (US); Barbara VERGANI, Macherio (IT); Edoardo CELLUPICA, Cernusco sul Naviglio (IT); Gianluca CAPRINI, Somma Lombardo (IT); Paola CORDELLA, Senago (IT); Gianluca FOSSATI, Milano (IT); IIaria ROCCHIO, Sesto San Giovanni (IT); Giovanni SANDRONE, Novara (IT); Andrea STEVENAZZI, Milano (IT); Christian STEINKÜHLER, Roma (IT)

(21) Appl. No.: 19/101,916

(22) PCT Filed: Aug. 7, 2023
(86) PCT No.: PCT/EP2023/071802

§ 371 Date: Feb. 7, 2025

(30) Foreign Application Priority Data
Aug. 8, 2022 (IT) 102022000016923

(51) Int. Cl. C07D 471/04; A61K 31/4192; A61K 31/428; A61K 31/4439; A61K 31/444; C07D 249/06; C07D 401/04; C07D 401/06; C07D 401/14; C07D 403/04; C07D 403/12; C07D 409/14; C07D 417/04; C07D 417/14

(52) U.S. Cl. CPC C07D 471/04; A61K 31/4192; A61K 31/428; A61K 31/4439; A61K 31/444; C07D 249/06; C07D 401/04; C07D 401/06; C07D 401/14; C07D 403/04; C07D 403/12; C07D 409/14; C07D 417/04; C07D 417/14

(57) ABSTRACT

The present invention relates to acylhydrazides obtained in situ by enzymatic hydrolysis of the parent prodrug 2-(difluoromethyl)- or 2-(trifluoromethyl)-1,3,4-oxadiazole, in histone deacetylase 6 (HDAC6).

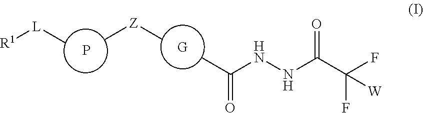

### FIELD OF THE INVENTION

The present invention relates to acylhydrazides obtained in situ by enzymatic hydrolysis of the parent prodrug 2-(difluoromethyl)- or 2-(trifluoromethyl)-1,3,4-oxadiazole, in histone deacetylase 6 (HDAC6).

### STATE OF THE ART OF THE INVENTION

Zn-dependent histone deacetylases (HDACs) are a family of 11 evolutionarily related hydrolases that catalyze the removal of acetyl or myristoyl residues from histones, non-histone proteins and polyamines (Biochemistry. 57, 3105-3114; Mol. Cell. Proteomics. 21, 100193; J. Genet. Genomics. 44, 243-250). Given the involvement of HDACs in numerous diseases, the pharmaceutical industry is pursuing the development of HDAC inhibitors (HDACis) since almost two decades, an effort that led to the approval of 5 molecules for the treatment of cancer (Br. J. Clin. Pharmacol. 87, 4577-4597). Unfortunately, the therapeutic benefit of HDACis has been limited by side effects due to the poor selectivity of these first generation molecules, that inhibit several to all of the Zn-dependent HDAC family members, thus also affecting crucial physiological functions.

HDAC6 stands out among the 11 human isoforms of Zn-dependent HDACs, as being the only one that has two homologous tandem catalytic domains (CD1 and CD2), and a zinc finger ubiquitin-binding domain. Furthermore, HDAC6 is mainly localized in the cytoplasm and its main substrates are not histones, but various non histone proteins such as α-tubulin, Foxp3, Hsp90, β-catenin, cortactin and peroxiredoxins. Intriguingly, HDAC6 KO mice are viable and fertile and do not show overt physiological dysfunctions (Mol. Cell. Biol. 28, 1688-1701). Also, selective HDAC6 inhibition was demonstrated to be well tolerated in both preclinical species and in human clinical trials (Oncologist. 26, 184-e366).

Since HDAC6 plays a role in both the ubiquitin-proteasome and the aggresome pathways, in regulating immune response, in the development of neuropathies and in Alzheimer's disease there is a lot of interest in the identification of highly specific HDAC6 inhibitors (J. Med. Chem. 64, 1362-1391).

The classical HDACi pharmacophore consists of a zinc binding group (ZBG), interacting with the active site Zn ion, a cap, which interacts with the outer region of the enzyme, and a linker that connects the ZBG to the cap. Most HDACis have a hydroxamic acid moiety as ZBG, a chemical group that has inherent stability and safety issues. The hydroxamate group is a metabolic hotspot that is associated with suboptimal pharmacokinetics and potential genotoxicity.

WO2022/029041, WO2022/013728, WO2021/127643, WO2020/212479 and WO2022/049496 disclose oxadiazole-based HDAC6 inhibitors as a potential alternative to hydroxamates but their mechanism of action is unclear.

### SUMMARY OF THE INVENTION

The present inventors identified difluoro- and trifluoro-acetyl hydrazides as tight binding inhibitors of HDAC6, forming long-lived complexes with the enzyme when formed in situ from suitably designed prodrugs.

The present inventors surprisingly found that difluoromethyl-1,3,4-oxiadiazoles (DFMOs) and trifluoromethyl-1,3,4-oxiadiazoles (TFMOs) are substrates of HDAC6, which is able to hydrolyse them to the corresponding difluoro- or trifluoro-acetyl hydrazides. DFMOs and TFMOs were hence selected as prodrugs for the in situ generation of acylhydrazides.

Using pre-steady state kinetics, rapid chromatography and mass spectrometry the present inventors dissected the mechanism of inhibition of DFMO and TFMO containing compounds, showing that HDAC6-catalyzed DFMO and TFMO ring hydration/opening leads to a tight and long-lived complex of the enzyme with the corresponding acylhydrazide. Once dissociated from the enzyme, the hydrated inhibitor may eventually reassociate with HDAC6, thereby undergoing further hydrolysis to yield a lower affinity hydrazide-complex.

The present inventors were also able to confirm this mechanism by solving the x-ray crystal structure of HDAC6-CD2 bound to a DFMO compound. The present inventors surprisingly found that the electron density in the active site was not compatible with the structure of the parent prodrug, but could perfectly fit with the structure of a hydrazide (i.e. the final hydrolysis product).

The acyl hydrazides containing compounds, object of the present invention, are not active if directly administered to the enzyme or to the cells, but they are impressively potent and selective when formed in situ from DFMO and TFMO prodrugs.

Focusing on the prodrugs (DFMO and TFMO containing HDAC6 inhibitors) the present inventors have been able to identify molecules with more than 10000-fold selectivity for HDAC6 over all other HDACs and excellent drug-like properties, safety features and pharmacologic activity in vitro and in vivo.

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

The term “halogen” refers herein to fluorine (F), chlorine (Cl), bromine (Br), or iodine (I).

The term “C₁-C₆ alkyl” herein refers to a branched or linear hydrocarbon containing from 1 to 6 carbon atoms. Examples of C₁-C₆ alkyl groups include but are not limited to methyl, ethyl, n-propyl, isopropyl, n-butyl, sec-butyl, isobutyl, tert-butyl, n-pentyl, n-hexyl.

The term “aryl” refers herein to mono- and poly-carbocyclic aromatic ring systems (i), wherein individual carbocyclic rings in the poly-carbocyclic ring systems may be fused or attached to each other by a single bond. Suitable aryl groups include, but are not limited to, phenyl, naphthyl and biphenyl.

The term “aryloxy” refers herein to O-aryl group, wherein “aryl” is as defined above.

The term “alkoxy” refers herein to O-alkyl group, wherein “alkyl” is as defined above.

The term “thioalkoxy” refers herein to S-alkyl group, wherein “alkyl” is as defined above. A preferred thioalkoxy group is thioethoxy (—SEt) or thiomethoxy (—SMe), and even more preferably it is thiomethoxy. In a different embodiment, the thioalkoxy group refers to an alkyl group wherein one of the nonterminal hydrocarbon units of the alkyl chain is replaced by a sulfur atom.

The term “halogenated” refers herein to halogen substitution, in other words, any of the above alkyl, alkoxy, thioalkoxy groups may be fully or partially substituted with a halogen atom. Preferably, the halogen atom is F or Cl, and more preferably it is F.

The term “cycloalkyl” refers herein to a saturated or unsaturated hydrocarbon ring, preferably having 3 to 10 carbon atoms. Examples of cycloalkyl include cyclopropyl, cyclobutyl, cyclopentyl, cyclohexyl, cycloheptyl and cyclooctyl.

The term “arylalkyl” refers herein to an aryl radical as defined herein, attached to an alkyl radical as defined herein. An example of arylalkyl is benzyl.

The term “deuterated” refers herein to deuterium substitution, in other words, the hydrogen atoms can be partially or fully replaced by deuterium.

The term “heterocycle” refers herein to a 4-, 5-, 6-, 7- or 8-membered monocyclic ring which is saturated or unsaturated and consisting of carbon atoms and one or more heteroatoms selected from N, O and S, and wherein the nitrogen and sulphur heteroatoms may optionally be oxidized and the nitrogen heteroatom can be optionally quaternized. The heterocyclic ring may be attached to any heteroatom or carbon atom, provided that the attachment results in the creation of a stable structure. The term also includes any bicyclic system wherein any of the above heterocyclic rings is fused to an aryl or another heterocycle. When the heterocyclic ring is an aromatic heterocyclic ring, it can be defined as a “heteroaromatic ring”.

The term “unsaturated ring” refers herein to a partially or completely unsaturated ring. For example, an unsaturated C6 monocyclic ring refers to cyclohexene, cyclohexadiene and benzene.

The term “substituted” refers herein to mono- or poly-substitution with a defined (or undefined) substituent provided that this single or multiple substitution is chemically allowed.

The term “physiologically acceptable excipient” herein refers to a substance devoid of any pharmacological effect of its own and which does not produce adverse reactions when administered to a mammal, preferably a human. Physiologically acceptable excipients are well known in the art and are disclosed, for instance in the Handbook of Pharmaceutical Excipients, sixth edition 2009, herein incorporated by reference.

The term “pharmaceutically acceptable salts or derivatives thereof” herein refers to those salts or derivatives which possess the biological effectiveness and properties of the salified or derivatized compound and which do not produce adverse reactions when administered to a mammal, preferably a human. The pharmaceutically acceptable salts may be inorganic or organic salts; examples of pharmaceutically acceptable salts include but are not limited to: carbonate, hydrochloride, hydrobromide, sulphate, hydrogen sulphate, citrate, maleate, fumarate, trifluoroacetate, 2-naphthalenesulphonate, and para-toluenesulphonate. Further information on pharmaceutically acceptable salts can be found in Handbook of pharmaceutical salts, P. Stahl, C. Wermuth, WILEY-VCH, 127-133, 2008, herein incorporated by reference. The pharmaceutically acceptable derivatives include the esters, the ethers and the N-oxides.

The terms “comprising”, “having”, “including” and “containing” are to be understood as open terms (meaning “including, but not limited to”) and are to be considered as a support also for terms such as “essentially consist of”, “essentially consisting of”, “consist of” or “consisting of”.

The terms “essentially consists of”, “essentially consisting of” are to be understood as semi-closed terms, meanings that no other ingredient affecting the novel characteristics of the invention is included (therefore optional excipients can be included).

The terms “consists of”, “consisting of” are to be understood as closed terms.

The term “isomers” refers to stereoisomers (or spatial isomers), i.e. diastereoisomers and enantiomers.

### DESCRIPTION OF THE FIGURES

FIG. 1 shows hypothesized mechanism for DFMO ring opening and related acyl-hydrazide and hydrazide formation.

FIG. 2 shows isolation of long-lived/tight complex (Example 10).

### DESCRIPTION OF THE INVENTION

Difluoromethyl-1,3,4-oxiadiazoles (DFMO) and trifluoromethyl-1,3,4-oxiadiazoles (TFMO) were found to be substrates of HDAC6, which is able to hydrolyse them to the corresponding difluoro- or trifluoro-acetyl hydrazides.

Kinetic studies were performed, incubating some DFMO and TFMO inhibitors with HDAC6 and analysing the medium by LC-MS. Indeed, the disappearing of the DFMO compounds and the formation of the corresponding acylhydrazides (used as standards for mass spectrometry studies) were observed. Using extreme, non-physiological conditions in terms of inhibitor and protein concentrations the formation of hydrazides was also observed. The same DFMO and TFMO inhibitors dissolved in the same buffer medium, for the same time, without enzyme did not show any degradation event. These experiments confirm that DFMO and TFMO can be considered substrates of HDAC6.

Intrigued by the slow binding/slow release kinetics associated to DFMO and TFMO compounds, spin column chromatography associated with LC-HRMS was used to identify the tight binding species, forming the long-lived inhibitor-HDAC6 complex (see example 10). When incubated with a DFMO or TFMO compound, the predominant species that coeluted with zHDAC6-CD2 was the corresponding acylhydrazide. This hydrate form and the corresponding hydrazide were also detected in the fractions containing free compounds, not bound to the enzyme. Interestingly, DFMO and TFMO parent compounds did not co-elute with the enzyme.

Intriguingly, the direct incubation of difluoromethylacylhydrazide with enzyme did not induce enzyme inhibition, suggesting that this species is active only when formed in situ. The present data instead suggest that the high-affinity species is the in situ formed acyl hydrazide.

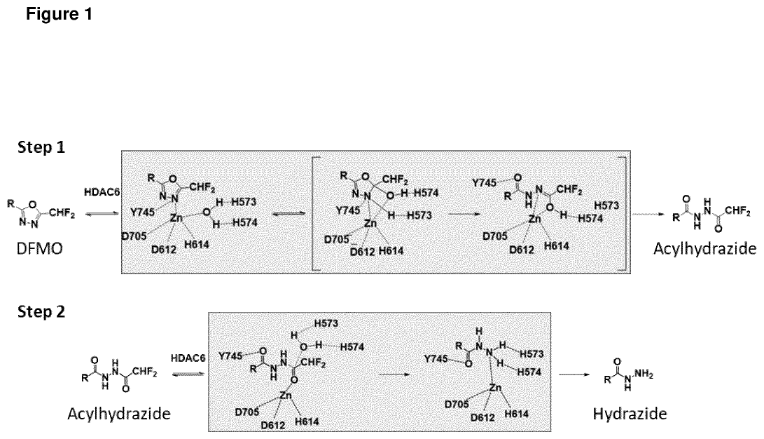

The crystallographic data, in combination with molecular modeling, supports the hypothesized mechanism. A hydrazide was found when co-crystalizing a DFMO containing compound. The postulated hydrazide could be formed as the result of two subsequent reactions. After entry of the inhibitor into the catalytic pocket, the active site Zn cation could enhance the electrophilic behavior of the sp² carbon atom directly bound to the CHF₂ group of the DFMO moiety, allowing a nucleophilic attack by the water molecule, whose presence in the metal cation coordination sphere is supported by modeling studies. The hydrated intermediate can further react in a ring-opening reaction to yield an acyl-hydrazide (FIG. 1, Step 1). In order to explain the hydrazide detected in the crystal structure an additional hydrolysis reaction has to be hypothesized which could be either enzyme catalyzed or take place in solution, upon release of the acyl-hydrazide from the enzyme active site. If the second hydrolysis reaction is indeed enzyme-catalyzed, the zinc coordination sphere needs to be restored by the entry of a second water molecule. The metal cation can then activate the acyl carbonyl, allowing the canonical deacetylation and the subsequent release of hydrazide and difluoroacetic acid (FIG. 1, Step 2).

The present inventors have surprisingly found that the DFMO inhibitors are hydrolyzed in the presence of HDAC6 to yield the corresponding difluoromethylacylhydrazide, which is the real active selective HDAC6 inhibitor.

According to a first aspect, the present invention relates to compounds of formula (I), pharmaceutically acceptable salts and isomers thereof:

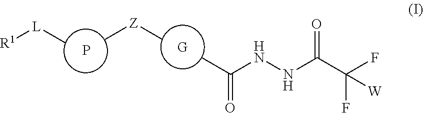

wherein:
W=H or F
G is a 5-membered heteroaromatic ring consisting of carbon atoms and 1 to 4 heteroatoms selected from N, O, S and Se, optionally substituted with C₁-C₃ alkyl, alkoxy, or thioalkoxy, halogenated derivatives thereof, or halogen, or hydroxy;
or G is the formula (Ia):

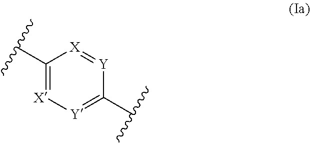

wherein X, X′, Y and Y′ are independently selected from CH, N, CF or CCl;
Z=CD₂-CD₂-, —CF₂—, —CHR²—, —NH—, —S—;
R²=H, halogen, C₁-C₆ alkyl or C₃-C₆ cycloalkyl, either unsubstituted or substituted with:
   hydroxy, carbonyl, C₁-C₃ alkoxy, aryloxy or thioalkoxy, or halogenated derivatives thereof;
   halogen;
   primary, secondary, or tertiary amine, substituted with C₁-C₆ alkyl, C₃-C₆ cycloalkyl or halogenated derivatives thereof;
   phenyl, pyridyl, thiophenyl, furan or pyrrole, either unsubstituted or substituted with C₁-C₃ alkyl, alkoxy, thioalkoxy or halogenated derivatives thereof, or halogen; or
   the following substructures or halogenated derivatives thereof:

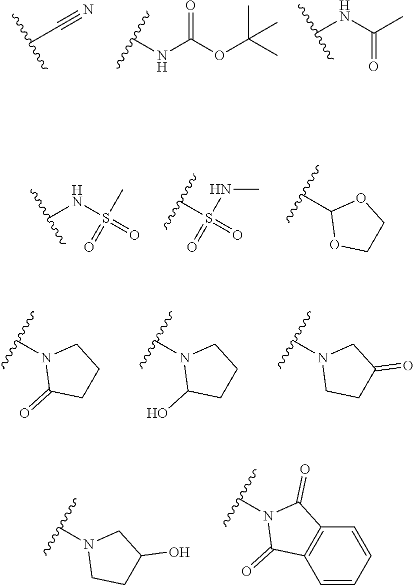

L is absent, C₁-C₆ alkyl, alkoxy or thioalkoxy, —(CH₂)ₘ—CHR⁴—(CH₂)ₒ—, —(CH₂)ₘ—CH(NHR⁴)—(CH₂)ₒ—, —(CH₂)ₘ—NR⁴—(CH₂)ₒ— or halogenated derivatives thereof;
wherein m and o are each independently 0, 1 or 2; or
L is selected among the following substructures (IIa)-(IIf) and halogenated derivatives thereof:

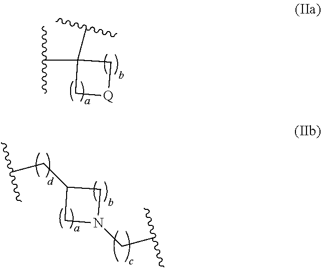

wherein a, b, c and d are independently 0, 1, 2, or 3 and a and b cannot be 0 at the same time;
Q is CH₂, NR⁴ or O;

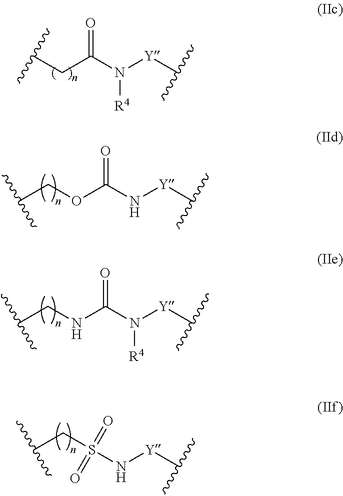

wherein n is 0, 1, or 2;
Y″ is absent, C₁-C₂ alkenyl, or is selected among the following substructures and halogenated derivatives thereof:

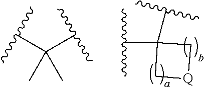

wherein a, b and Q are as defined above;
R⁴=H, C₁-C₄ alkyl unsubstituted or substituted with:
   halogen
   phenyl, pyridyl, thiophenyl, furan or pyrrole, either unsubstituted or substituted with C₁-C₃ alkyl, alkoxy, thioalkoxy or halogenated derivatives thereof, or halogen;
P is an unsubstituted or substituted, aromatic or non-aromatic 5 to 10 membered heterocyclic ring, wherein the ring consists of carbon atoms and one or more heteroatoms selected from N, O and S;
R¹ is absent, halogen, unsubstituted or substituted —C(═O)C₁-C₄ alkyl, C₁-C₄ alkyl, C₄-C₆ cycloalkenyl, C₆-C₁₂ aryl, or 5- to 9-membered heteroaryl including at least one heteroatom selected from N, O and S;
with the proviso that the following compounds are excluded:
3-[2-[[4-[[(2,2-difluoroacetyl)amino]carbamoyl]phenyl]methyl]tetrazol-5-yl]benzoic acid
2-((5-(6-aminopyridin-3-yl)-2H-tetrazol-2-yl)methyl)-N′-(2,2-difluoroacetyl)pyrimidine-5-carbohydrazide
tert-butyl (5-(1-((6-(2-(2,2-difluoroacetyl)hydrazine-1-carbonyl)pyridazin-3-yl)methyl)-1H-1,2,3-triazol-4-yl)pyridin-2-yl)carbamate
N′-(2,2-difluoroacetyl)-4-((5,5-dimethyl-2,4-dioxo-3-phenylimidazolidin-1-yl)methyl)-3-fluorobenzohydrazide
4-((5,5-dimethyl-2,4-dioxo-3-phenylimidazolidin-1-yl)methyl)-3-fluoro-N′-(2,2,2-trifluoroacetyl)benzohydrazide
N′-(2,2-difluoroacetyl)-4-((2,5-dioxo-3-phenylimidazolidin-1-yl)methyl)-3-fluorobenzohydrazide
N′-(2,2-difluoroacetyl)-6-((1-(3-fluorophenyl)-8-(oxetan-3-yl)-2,4-dioxo-1,3,8-triazaspiro[4.5]decan-3-yl)methyl)nicotinohydrazide
4-((1H-imidazol-1-yl)methyl)-N′-(2,2-difluoroacetyl)benzohydrazide
N′-(2,2-difluoroacetyl)-4-(pyrimidin-2-ylamino)benzohydrazide
benzyl 4-((5-(2-(2,2-difluoroacetyl)hydrazine-1-carbonyl)pyrimidin-2-yl)amino)-4-phenylpiperidine-1-carboxylate
4-((1-(3-chloro-4-cyanophenyl)-3,5-dimethyl-1H-pyrazol-4-yl)methyl)-N′-(2,2,2-trifluoroacetyl)benzohydrazide.

Another class of preferred compounds comprises compounds of formula (I), pharmaceutically acceptable salts and isomers thereof, wherein R² is H, or C₁-C₃ alkyl, either unsubstituted or substituted with:
C₁-C₃ alkyl or C₃-C₆ cycloalkyl
hydroxy, carbonyl, C₁-C₂ alkoxy, aryloxy or thioalkoxy, or halogenated derivatives thereof;
halogen;
primary, secondary, or tertiary amine, substituted with C₁-C₃ alkyl, C₃-C₆ cycloalkyl or halogenated derivatives thereof;
phenyl, pyridyl, thiophenyl, furan or pyrrole, either unsubstituted or substituted with C₁-C₃ alkyl, alkoxy, thioalkoxy or halogenated derivatives thereof, or halogen; or
the following substructures or halogenated derivatives thereof:

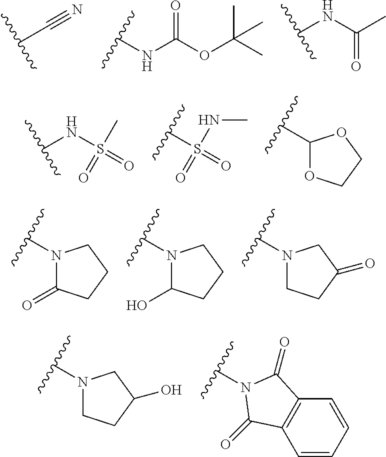

Another class of preferred compounds comprises compounds of formula (I), pharmaceutically acceptable salts and isomers thereof, wherein P is

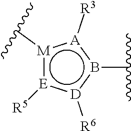

wherein:
A=C, N, O, S;
B=C, N;
D=C, N, O;
E=C, N, O;
M=C, N;
R⁵ and R⁶ are independently —H, halogen, ═O, C₁-C₆ alkyl, alkoxy or thioalkoxy, C₃-C₆ cycloalkyl, or halogenated derivatives thereof, optionally substituted with carbonyl or carboxy, or R⁵ and R⁶ are independently selected among the following substructures:

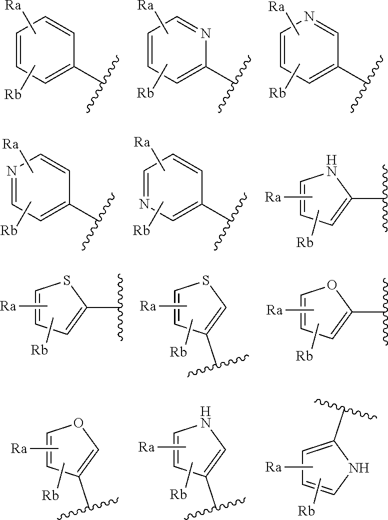

R³ is absent, —H, C₁-C₆ alkyl optionally substituted with —OH or —N(C₁-C₅ alkyl)₂, -LR¹ or is selected among the following substructures:

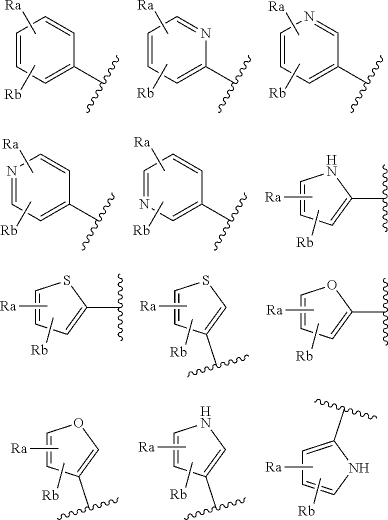

when R³=-LR¹, substitution on M is absent;
Ra and Rb are independently H, halogen, C₁-C₆ alkyl, alkoxy or thioalkoxy, C₃-C₆ cycloalkyl, or halogenated derivatives thereof.

Another class of preferred compounds comprises compounds of formula (I), pharmaceutically acceptable salts and isomers thereof, wherein P is selected among the following substructures:

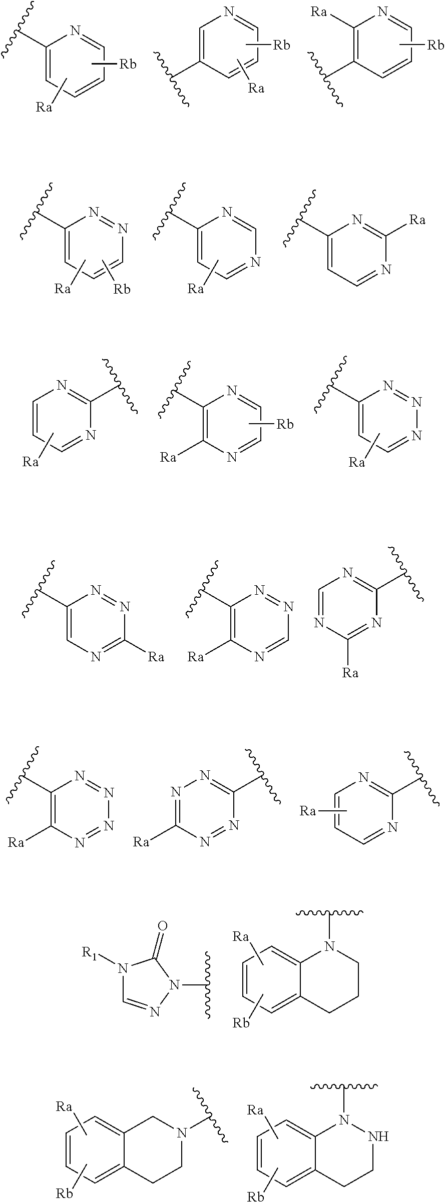

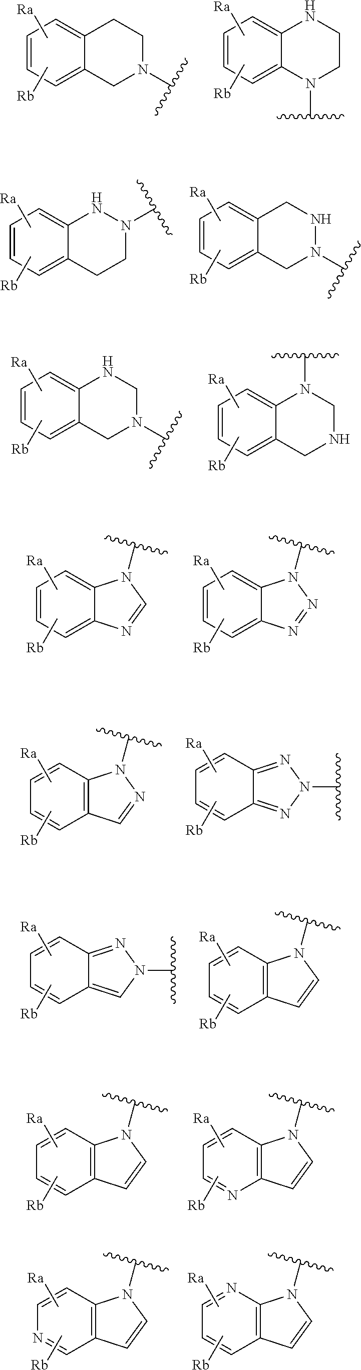

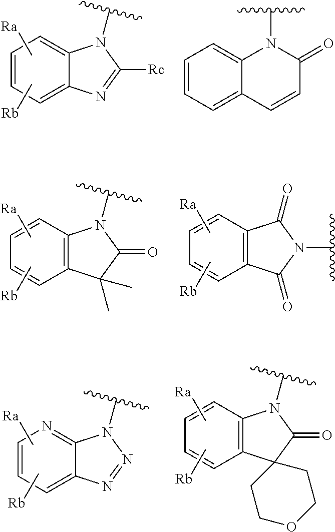

wherein Ra and Rb are as defined in claim 3 or are -L-R¹;
Rc is H, halogen, C₁-C₆ alkyl, alkoxy or thioalkoxy, C₃-C₆ cycloalkyl, or halogenated derivatives thereof, or —NH₂.

Another class of preferred compounds comprises compounds of formula (I), pharmaceutically acceptable salts and isomers thereof, wherein R¹ is selected among the following substructures:

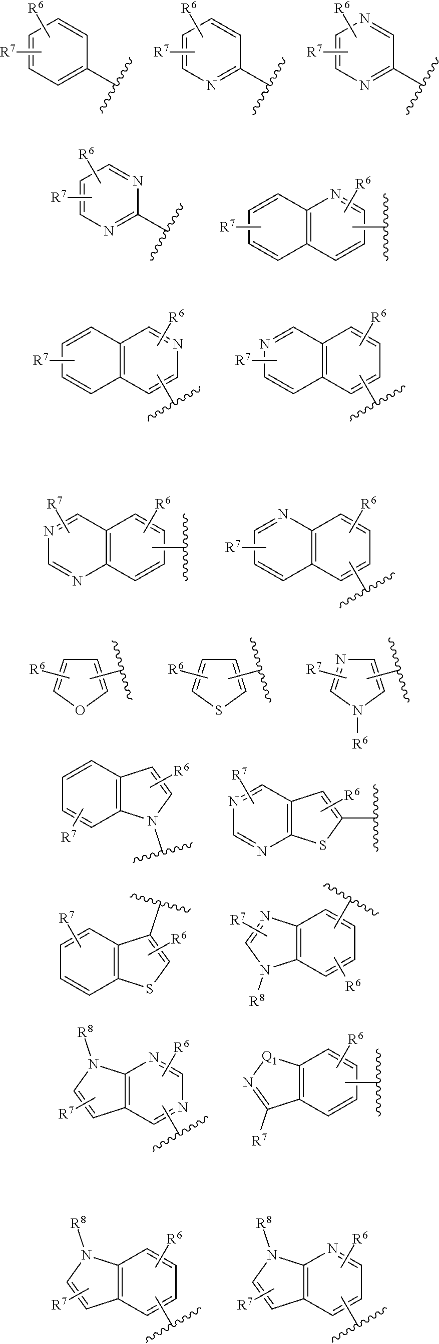

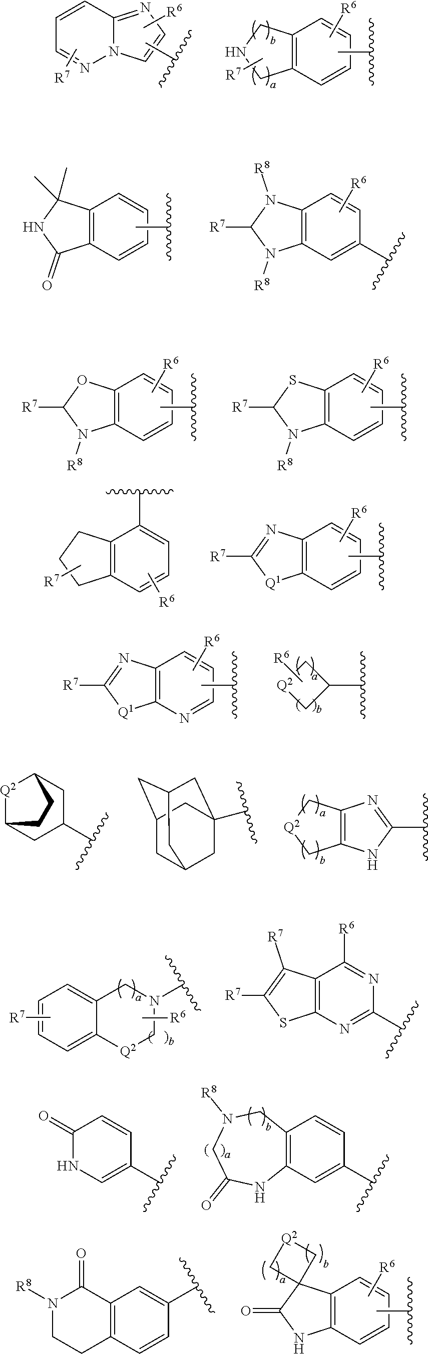

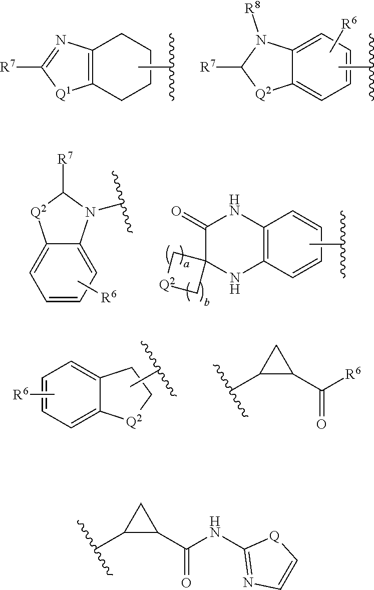

wherein R⁶ and R⁷ are independently selected from the group comprising: —H, -D, —OH, C₁-C₄ alkyl, alkoxy or thioalkoxy, C₃-C₆ cycloalkyl or halogenated derivatives thereof, halogen, —(CH₂)ₐNR′R″, —NHR⁸, —C(═O)OR′, —C(═O)R⁹, —C(═NH)R⁹, —NO₂, —CN, -Ph, —SO₂—N R′R″, ═O, ═NR⁸, —SO₂—C₁-C₄ alkyl, or
R⁶ and R⁷ are independently selected among the following substructures:

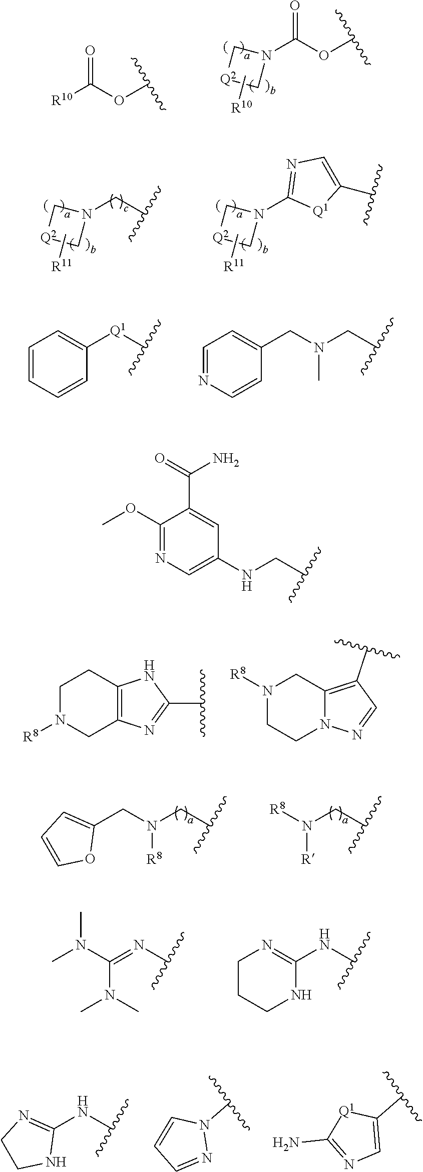

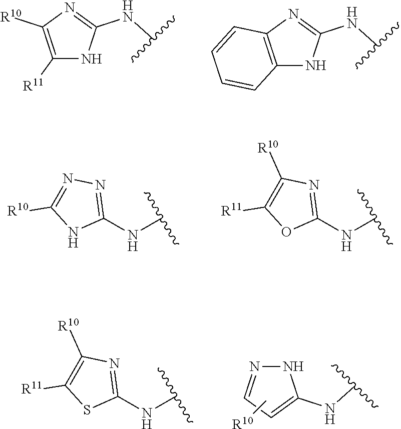

R⁸=—H, -D, —OH, C₁-C₆ alkyl, C₃-C₆ cycloalkyl or halogenated derivatives thereof, —(CH₂)ₐNR′R″—C(═O)OR′, —C(═O)R⁹, —C(═NH)R⁹, —(CH₂)ₐPh, —(CH₂)ₐPy, —SO₂—C₁-C₄ alkyl or R⁸ is selected among the following substructures:

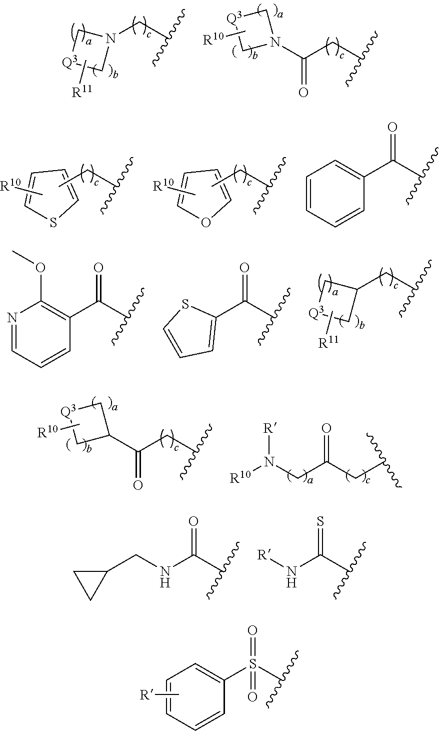

R⁹=—NR′R″, C₁-C₄ alkyl, or halogenated derivatives thereof or is selected among the following substructures:

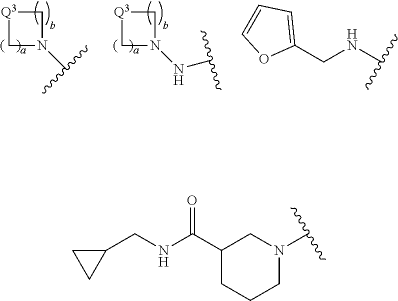

R¹⁰ and R¹¹ are independently selected from —H, C₁-C₄ alkyl, C₃-C₆ cycloalkyl or halogenated derivatives thereof, —OR′, —C(═O)OR′, —C(═O)R′, or halogen;
Q¹ is CH₂, O, S, NR⁸;
Q² and Q³ are independently CR′R″, CF₂, O, S, NR⁸;
R′ and R″ are independently —H, C₁-C₄ alkyl, C₃-C₆ cycloalkyl or halogenated derivatives thereof;
a, b, c, and R⁸ are as defined above.

Another class of preferred compounds comprises compounds of formula (I), pharmaceutically acceptable salts and isomers thereof, wherein R¹ is selected among the following substructures:

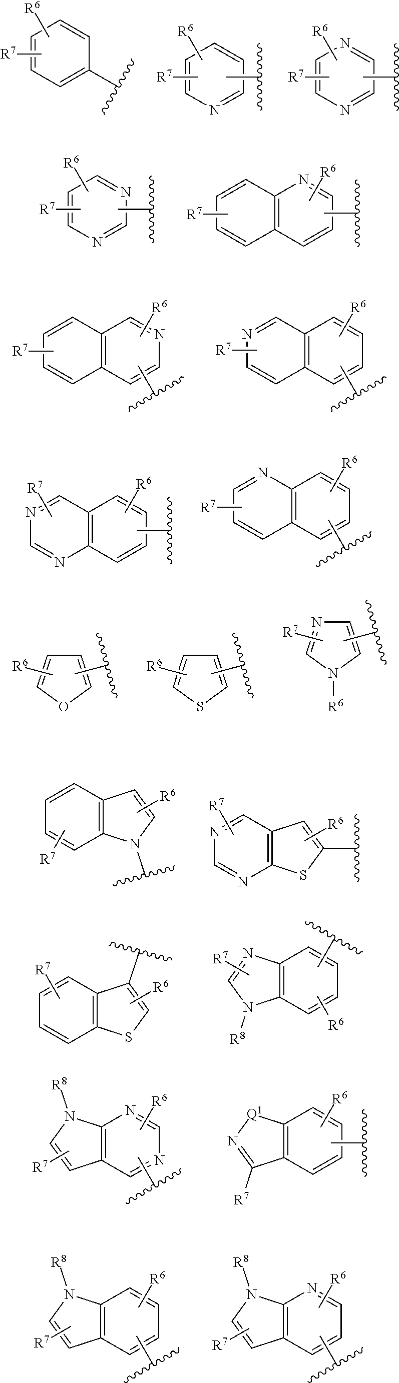

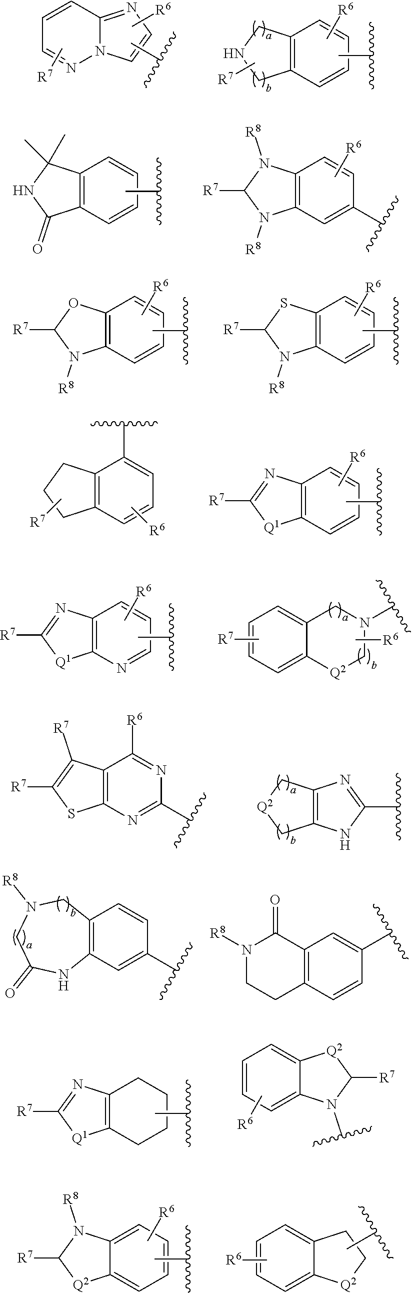

wherein R⁶ and R⁷ are independently selected from the group comprising: —H, -D, —OH, C₁-C₄ alkyl, alkoxy or thioalkoxy, C₃-C₆ cycloalkyl or halogenated derivatives thereof, halogen, —(CH₂)ₐNR′R″, —NHR⁸, —C(═O)R⁹, —NO₂, -Ph, —SO₂—NR′R″, ═O, ═NR⁸, —SO₂—C₁-C₄ alkyl, or are independently among the following substructures:

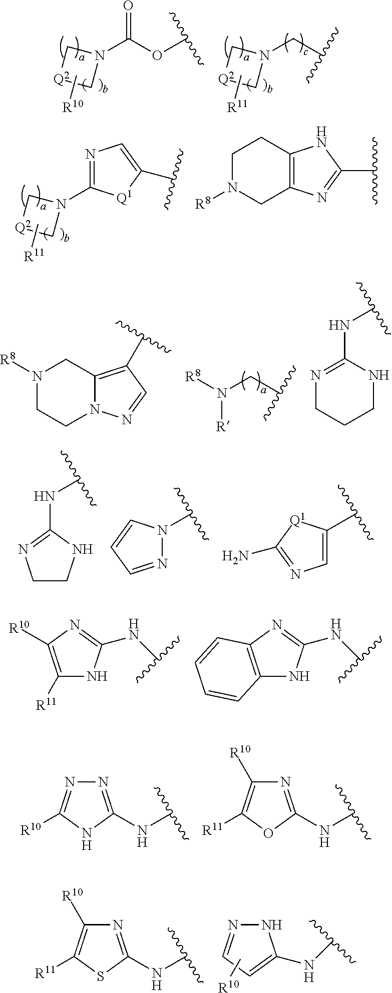

R⁸=—H, -D, —OH, C₁-C₆ alkyl, C₃-C₆ cycloalkyl or halogenated derivatives thereof, —(CH₂)ₐNR′R″, —C(═O)OR′, —C(═O)R⁹, —C(═NH)R⁹, —SO₂—C₁-C₄ alkyl or R⁸ is selected among the following substructures:

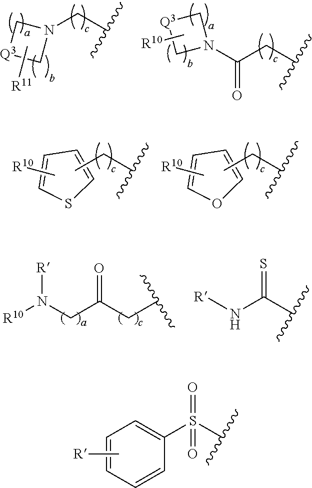

R⁹=—NR′R″, C₁-C₄ alkyl, or halogenated derivatives thereof or is selected among the following substructures:

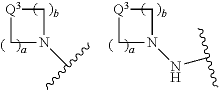

R¹⁰ and R¹¹ are independently selected from —H, C₁-C₄ alkyl, C₃-C₆ cycloalkyl or halogenated derivatives thereof, —OR′, —C(═O)OR′, —C(═O)R′, or halogen;
Q¹ is CH₂, O, S, NR⁸;
Q² and Q³ are independently CR′R″, CF₂, O, S, NR⁸;
R′ and R″ are independently —H, C₁-C₄ alkyl, C₃-C₆ cycloalkyl or halogenated derivatives thereof;
a, b, c, and R⁸ are as defined above.

Another class of preferred compounds comprises compounds of formula (I), pharmaceutically acceptable salts and isomers thereof, wherein R¹ is selected among the following substructures:

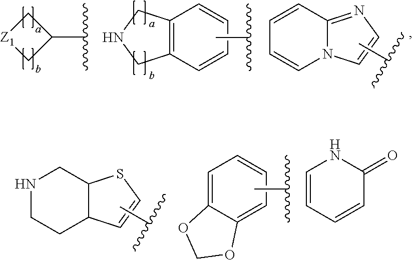

wherein a, b are independently 0, 1, 2, or 3 and a and b cannot be 0 at the same time;
Z₁ is CH₂, NH, or O;
and wherein at least one H of R¹ is optionally substituted with: halogen, —(CH₂)ₙ-Q4-Q5-Rd;
n=0, 1 or 2;
Q4=absent, —SO₂—, —NH—, —N(C₁-C₅ alkyl)-, —NHC(═O)—, —N(C₁-C₅ alkyl)C(═O)— or —C(═O)—;
Q5=absent, C₁-C₅ alkylene, —NH—, —(C₁-C₅ alkylene)-NH—C(═O)— or —N(C₁-C₅alkyl);
Rd=—OH, C₁-C₅ alkyl, C₁-C₅ haloalkyl, —NR′R″, C₁-C₅ alkoxy, 5 or 6 membered heteroaryl including 1 to 3 N,

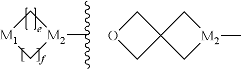

wherein e and f are independently 1 or 2;
M1 is CH₂, O, NH or SO₂, M₂ is CH or N and wherein at least one H of Rd is optionally substituted with OH, halogen, C₁-C₅ alkyl, C₁-C₅ haloalkyl, —C(═O)—(C₁-C₅ alkyl), —C(═O)O(C₁-C₅alkyl); —NH—C(═O)—O(C₁-C₅ alkyl), —NR′R″,

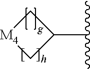

wherein g and h are independently 0, or 1, but cannot be 0 at the same time;
M₄ is CH₂, 0, NH and at least one H of M4 is optionally substituted with halogen, C₁-C₅ alkyl, C₃-C₆ cycloalkyl or —C(═O)—O(C₁-C₅ alkyl);
R′ and R″ are independently —H or C₁-C₄ alkyl.

The following compounds of formula (I) are preferred:
N′-(2,2-difluoroacetyl)-4-((4-(4-((4,5-dihydro-1H-imidazol-2-yl)amino)phenyl)-1H-1,2,3-triazol-1-yl)methyl)benzohydrazide (comp. 1)
4-((4-(2-aminobenzo[d]thiazol-6-yl)-1H-1,2,3-triazol-1-yl)methyl)-N′-(2,2-difluoroacetyl)-3-fluorobenzohydrazide (comp. 2)
N′-(2,2-difluoroacetyl)-6-((4-(p-tolyl)-1H-1,2,3-triazol-1-yl)methyl)nicotinohydrazide (comp. 8)
N′-(2,2-difluoroacetyl)-6-((4-(m-tolyl)-1H-1,2,3-triazol-1-yl)methyl)nicotinohydrazide (comp. 9)
N-(3-(1-(4-(2-(2,2-difluoroacetyl)hydrazine-1-carbonyl)-2-fluorobenzyl)-1H-1,2,3-triazol-4-yl)phenyl)acetamide (comp. 12)
N′-(2,2-difluoroacetyl)-6-((4-phenyl-1H-1,2,3-triazol-1-yl)methyl)nicotinohydrazide (comp. 13)
N′-(2,2-difluoroacetyl)-6-((4-(2-fluorophenyl)-1H-1,2,3-triazol-1-yl)methyl)nicotinohydrazide (comp. 14)
N′-(2,2-difluoroacetyl)-5-fluoro-6-((4-(pyridin-2-yl)-1H-1,2,3-triazol-1-yl)methyl)nicotinohydrazide (comp. 15)
N′-(2,2-difluoroacetyl)-6-((5-phenyl-2H-tetrazol-2-yl)methyl)nicotinohydrazide (comp. 16)
N′-(2,2-difluoroacetyl)-6-((5-(thiophen-2-yl)-2H-tetrazol-2-yl)methyl)nicotinohydrazide (comp. 17)
N′-(2,2-difluoroacetyl)-6-((4-(3-fluorophenyl)-1H-1,2,3-triazol-1-yl)methyl)nicotinohydrazide (comp. 18)
N′-(2,2-difluoroacetyl)-6-((4-(thiophen-2-yl)-1H-1,2,3-triazol-1-yl)methyl)nicotinohydrazide (comp. 19)
N′-(2,2-difluoroacetyl)-6-((4-(pyridin-2-yl)-1H-1,2,3-triazol-1-yl)methyl)nicotinohydrazide (comp. 20)
6-((4-(1H-pyrrolo[2,3-b]pyridin-5-yl)-1H-1,2,3-triazol-1-yl)methyl)-N′-(2,2-difluoroacetyl)nicotinohydrazide (comp. 27)
4-(1-(4-(2-aminobenzo[d]thiazol-6-yl)-1H-1,2,3-triazol-1-yl)ethyl)-N′-(2,2-difluoroacetyl)-3-fluorobenzohydrazide (comp. 30)
4-((4-(2-aminobenzo[d]thiazol-6-yl)-1H-1,2,3-triazol-1-yl)methyl)-2-chloro-N′-(2,2-difluoroacetyl)benzohydrazide (comp. 31)
4-((4-(2-aminobenzo[d]thiazol-6-yl)-1H-1,2,3-triazol-1-yl)methyl)-N′-(2,2-difluoroacetyl)benzohydrazide (comp. 32)
6-((4-(1H-indazol-4-yl)-1H-1,2,3-triazol-1-yl)methyl)-N′-(2,2-difluoroacetyl)nicotinohydrazide (comp. 34)
4-((4-(1H-indol-4-yl)-1H-1,2,3-triazol-1-yl)methyl)-N′-(2,2-difluoroacetyl)-3-fluorobenzohydrazide (comp. 36)
4-((4-(2-aminobenzo[d]thiazol-6-yl)-1H-1,2,3-triazol-1-yl)methyl)-3-chloro-N′-(2,2-difluoroacetyl)benzohydrazide (comp. 37)
6-((4-(2-aminobenzo[d]thiazol-6-yl)-1H-1,2,3-triazol-1-yl)methyl)-N′-(2,2-difluoroacetyl)nicotinohydrazide (comp. 38)
6-(1-(4-(6-aminopyridin-3-yl)-1H-1,2,3-triazol-1-yl)ethyl)-N′-(2,2-difluoroacetyl)nicotinohydrazide (comp. 39)
6-((4-(1H-indazol-4-yl)-1H-1,2,3-triazol-1-yl)methyl)-N′-(2,2-difluoroacetyl)-5-fluoronicotinohydrazide (comp. 40)
4-((4-(6-aminopyridin-3-yl)-1H-1,2,3-triazol-1-yl)methyl)-3,5-difluoro-N′-(2,2,2-trifluoroacetyl)benzohydrazide (comp. 42)
4-((4-(6-aminopyridin-3-yl)-1H-1,2,3-triazol-1-yl)methyl)-3-fluoro-N′-(2,2,2-trifluoroacetyl)benzohydrazide (comp. 43)
4-((4-(6-aminopyridin-3-yl)-1H-1,2,3-triazol-1-yl)methyl)-2-fluoro-N′-(2,2,2-trifluoroacetyl)benzohydrazide (comp. 44)
4-((4-(6-aminopyridin-3-yl)-1H-1,2,3-triazol-1-yl)methyl)-N′-(2,2-difluoroacetyl)benzohydrazide (comp. 45)
4-((4-(6-aminopyridin-3-yl)-1H-1,2,3-triazol-1-yl)methyl)-N′-(2,2-difluoroacetyl)-3,5-difluorobenzohydrazide (comp. 46)
4-((4-(6-aminopyridin-3-yl)-1H-1,2,3-triazol-1-yl)methyl)-N′-(2,2-difluoroacetyl)-3-fluorobenzohydrazide (comp. 47)
5-[[4-(2-amino-1,3-benzothiazol-6-yl)triazol-1-yl]methyl]-N′-(2,2-difluoroacetyl)thiophene-2-carbohydrazide (comp. 48)
5-[[4-(6-aminopyridin-3-yl)triazol-1-yl]methyl]-N′-(2,2-difluoroacetyl)thiophene-2-carbohydrazide (comp. 49).

| Comp. | Structure |
|---|---|
| 1 | 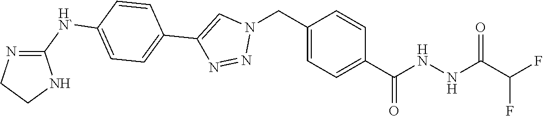 |
| 2 | 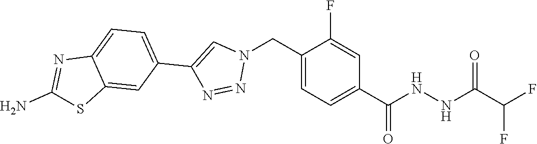 [SMILES: Nc1nc2ccc(-c3cn(Cc4ccc(C(=O)NNC(=O)C(F)F)cc4F)nn3)cc2s1] |
| 8 | 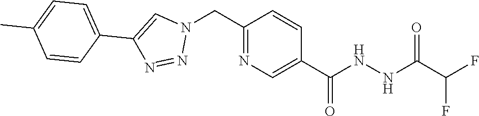 [SMILES: Cc1ccc(-c2cn(Cc3ccc(C(=O)NNC(=O)C(F)F)cn3)nn2)cc1] |
| 9 | 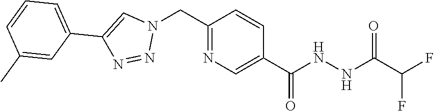 [SMILES: Cc1cccc(-c2cn(Cc3ccc(C(=O)NNC(=O)C(F)F)cn3)nn2)c1] |
| 12 | 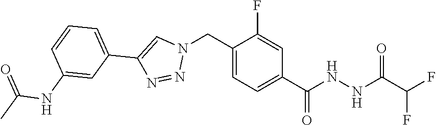 [SMILES: CC(=O)Nc1cccc(-c2cn(Cc3ccc(C(=O)NNC(=O)C(F)F)cc3F)nn2)c1] |
| 13 | 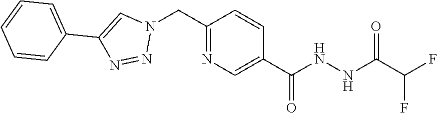 [SMILES: O=C(NNC(=O)C(F)F)c1ccc(Cn2cc(-c3ccccc3)nn2)nc1] |
| 14 | 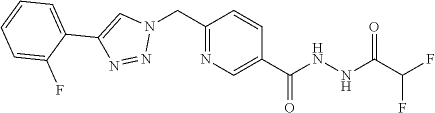 [SMILES: O=C(NNC(=O)C(F)F)c1ccc(Cn2cc(-c3ccccc3F)nn2)nc1] |
| 15 | 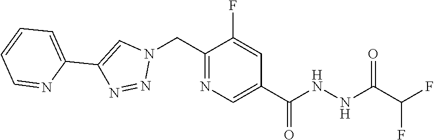 [SMILES: O=C(NNC(=O)C(F)F)c1cnc(Cn2cc(-c3ccccn3)nn2)c(F)c1] |
| 16 | 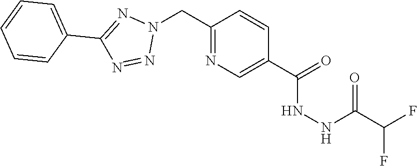 [SMILES: O=C(NNC(=O)C(F)F)c1ccc(Cn2nnc(-c3ccccc3)n2)nc1] |
| 17 | 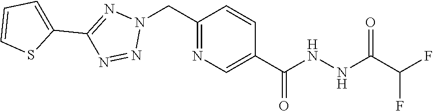 [SMILES: O=C(NNC(=O)C(F)F)c1ccc(Cn2nnc(-c3cccs3)n2)nc1] |
| 18 | 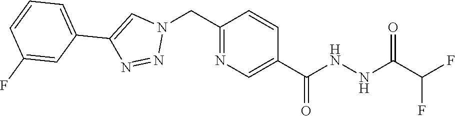 [SMILES: O=C(NNC(=O)C(F)F)c1ccc(Cn2cc(-c3cccc(F)c3)nn2)nc1] |
| 19 | 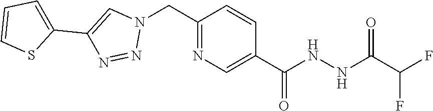 [SMILES: O=C(NNC(=O)C(F)F)c1ccc(Cn2cc(-c3cccs3)nn2)nc1] |
| 20 | 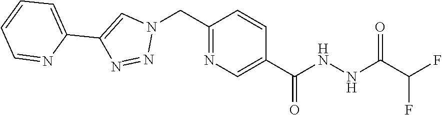 [SMILES: O=C(NNC(=O)C(F)F)c1ccc(Cn2cc(-c3ccccn3)nn2)nc1] |
| 27 | 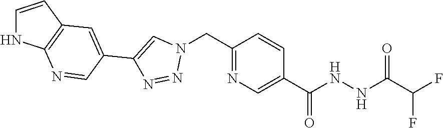 [SMILES: O=C(NNC(=O)C(F)F)c1ccc(Cn2cc(-c3cnc4[nH]ccc4c3)nn2)nc1] |
| 30 | 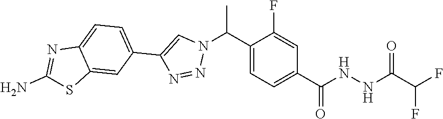 [SMILES: CC(c1ccc(C(=O)NNC(=O)C(F)F)cc1F)n1cc(-c2ccc3nc(N)sc3c2)nn1] |
| 31 | 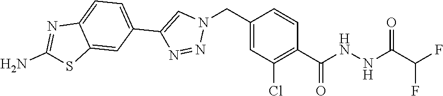 [SMILES: Nc1nc2ccc(-c3cn(Cc4ccc(C(=O)NNC(=O)C(F)F)c(Cl)c4)nn3)cc2s1] |
| 32 | 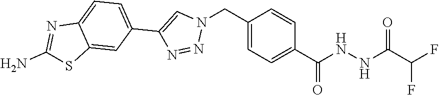 [SMILES: Nc1nc2ccc(-c3cn(Cc4ccc(C(=O)NNC(=O)C(F)F)cc4)nn3)cc2s1] |
| 34 | 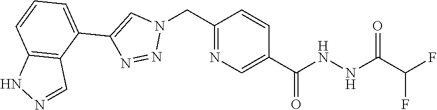 [SMILES: O=C(NNC(=O)C(F)F)c1ccc(Cn2cc(-c3cccc4[nH]ncc34)nn2)nc1] |
| 36 | 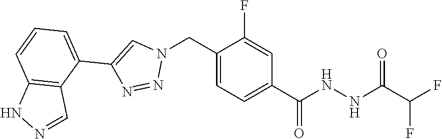 [SMILES: O=C(NNC(=O)C(F)F)c1ccc(Cn2cc(-c3cccc4[nH]ncc34)nn2)c(F)c1] |
| 37 | 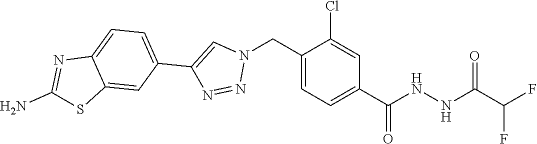 [SMILES: Nc1nc2ccc(-c3cn(Cc4ccc(C(=O)NNC(=O)C(F)F)cc4Cl)nn3)cc2s1] |
| 38 | 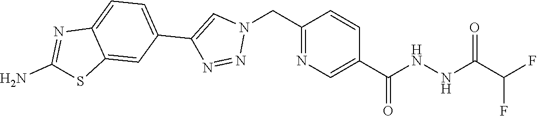 [SMILES: Nc1nc2ccc(-c3cn(Cc4ccc(C(=O)NNC(=O)C(F)F)cn4)nn3)cc2s1] |
| 39 | 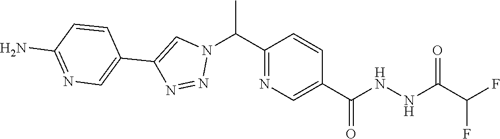 [SMILES: CC(c1ccc(C(=O)NNC(=O)C(F)F)cn1)n1cc(-c2ccc(N)nc2)nn1] |
| 40 | 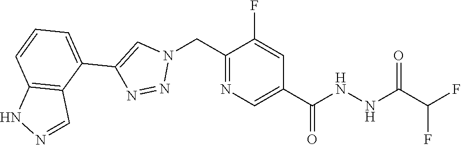 [SMILES: O=C(NNC(=O)C(F)F)c1cnc(Cn2cc(-c3cccc4[nH]ncc34)nn2)c(F)c1] |
| 42 | 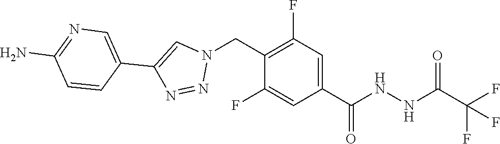 [SMILES: Nc1ccc(-c2cn(Cc3c(F)cc(C(=O)NNC(=O)C(F)(F)F)cc3F)nn2)cn1] |
| 43 | 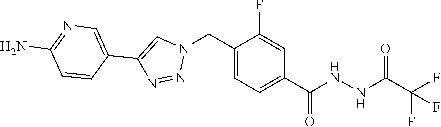 [SMILES: Nc1ccc(-c2cn(Cc3ccc(C(=O)NNC(=O)C(F)(F)F)cc3F)nn2)cn1] |
| 44 | 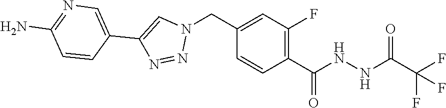 [SMILES: Nc1ccc(-c2cn(Cc3ccc(C(=O)NNC(=O)C(F)(F)F)c(F)c3)nn2)cn1] |
| 45 | 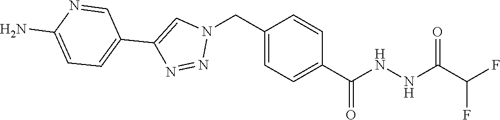 [SMILES: Nc1ccc(-c2cn(Cc3ccc(C(=O)NNC(=O)C(F)F)cc3)nn2)cn1] |
| 46 | 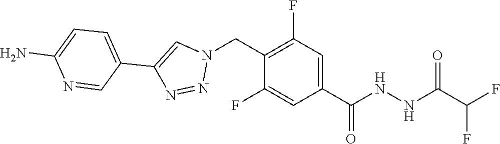 [SMILES: Nc1ccc(-c2cn(Cc3c(F)cc(C(=O)NNC(=O)C(F)F)cc3F)nn2)cn1] |
| 47 | 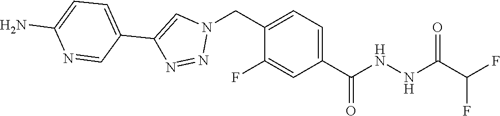 [SMILES: Nc1ccc(-c2cn(Cc3ccc(C(=O)NNC(=O)C(F)F)cc3F)nn2)cn1] |
| 48 | 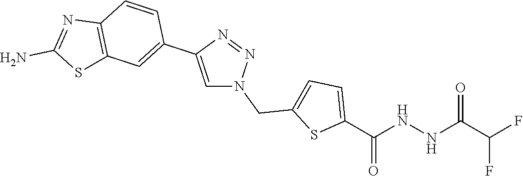 [SMILES: Nc1nc2ccc(-c3cn(Cc4ccc(C(=O)NNC(=O)C(F)F)s4)nn3)cc2s1] |
| 49 | 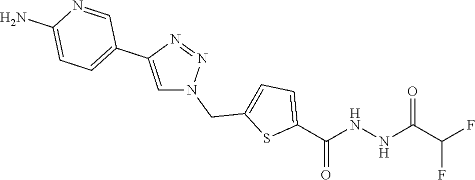 [SMILES: Nc1ccc(-c2cn(Cc3ccc(C(=O)NNC(=O)C(F)F)s3)nn2)cn1] |

Compounds of the present invention may contain one or more chiral centres (asymmetric carbon atoms), therefore they may exist in enantiomeric and/or diastereoisomeric forms.

All possible optical isomers, alone or in a mixture with each other, fall within the scope of the present invention.

A second object of the present invention are the above compounds of formula (I) for use as medicaments.

A third object of the present invention are the above compounds for use in the prevention and/or treatment of a disease or disorder modulated by HDAC6.

The compounds of the invention are preferably useful for the treatment of peripheral neuropathies, both genetically originated, such as, for example, but not limited to, Charcot-Marie-Tooth disease, medication induced (chemotherapy or antibiotics, such as metronidazole and fluoroquinolone classes) and due to systemic diseases, such as diabetes or leprosy or in general for the treatment of peripheral neuropathies correlated to severe axonal transport deficit. The compounds of invention can also be useful for treatment of chemotherapy-related cognitive impairment (CRCI).

The compounds of the invention are preferably useful for the treatment of graft rejection, GVHD, myositis, diseases associated with abnormal lymphocyte functions, multiple myeloma, non-Hodgkin lymphoma, peripheral neuropathy, autoimmune diseases, inflammatory diseases, cancer and neurodegenerative diseases, ocular diseases (e.g. uveitis).

### General Synthetic Pathway

The compounds described in the present invention can be prepared by using methods known to those skilled in the art.

All starting materials, reagents, acids, bases, solvents and catalysts used in the synthesis of the described compounds are commercially available.

Reaction progression was monitored by TLC, HPLC, UPLC or HPLC-MS analysis.

Acylhydrazides are obtained in situ by enzymatic hydrolysis of the parent prodrug 2-(difluoromethyl)- or 2-(trifluoromethyl)-1,3,4-oxadiazole, in histone deacetylase 6 (HDAC6).

Similarly, thou less efficiently, 2-(difluoromethyl)- and 2-(trifluoromethyl)-1,3,4-oxadiazoles can be hydrolyzed in aqueous acidic (TFA) or basic (LiOH or methanolic ammonia) conditions, generating difluoro- and trifluoro-acetyl hydrazides, respectively.

2-(difluoromethyl)- and 2-(trifluoromethyl)-1,3,4-oxadiazole moieties were synthesized as described in the literature (Marchini, M. et al, 2021, “2-(4-((5-(benzo[b]thiophen-3-yl)-1H-tetrazol-1-yl)methyl)phenyl)-5-(difluoromethyl)-1,3,4-oxadiazole derivatives and similar compounds as selective inhibitors of histone deacetylase 6 (HDAC6) for use in treating e.g. peripheral neuropathy”, WO2022029041; Lee, J. K. et al, “Novel compounds as histone deacetylase 6 inhibitor, and pharmaceutical composition comprising the same”, WO2022013728; Vara Salazar, Y. I. et al, 2020, “1,3,4-oxadiazole derivatives as histone deacetylase inhibitors”, WO2020212479; Yates, C., 2018, “Metalloenzyme inhibitor compounds” WO2018165520). In most of the cases the corresponding hydrazide was treated with an excess of difluoroacetic or trifluoroacetic anhydride, which acts both as an acylating and a dehydrating agent (Lee, J. et al, 2017; “1,3,4-Oxadiazole sulfonamide derivatives as histone deacetylase 6 inhibitors and their pharmaceutical composition and preparation”; WO2017018805). In some cases, 2-(difluoromethyl)- or 2-(trifluoromethyl)-1,3,4-oxadiazole moiety was prepared starting from the corresponding tetrazole, which was converted into 2-(difluoromethyl)- or 2-(trifluoromethyl)-1,3,4-oxadiazole in presence of difluoroacetic or trifluoroacetic anhydride (Vereshchagin et al Rus. J. Org. Chem. 2007, 43(11), 1710-1714). Moreover, an acylhydrazide can be converted to oxadiazole in the presence of other dehydrating reagents, such as Burgess' reagent (Lee, J. et al, “1,3,4-Oxadiazole derivative compounds as histone deacetylase 6 inhibitor, and the pharmaceutical composition comprising the same”, WO201723133), or tosyl chloride (Ito M. et al, 2019, “Heterocylic compound”, WO2019027054).

Other methods to prepare acylhydrazides include acylation of an hydrazide in the presence of almost stoichiometric amount of difluoroacetic or trifluoroacetic anhydride (Lee, J. et al, 2017; “1,3,4-Oxadiazole sulfonamide derivatives as histone deacetylase 6 inhibitors and their pharmaceutical composition and preparation”; WO2017018805), or functionalization of a carboxylic acid with difluoroacetyl or trifluoroacetyl hydrazide, using the common amide coupling procedures and reagents, i.e. via activated ester using HATU, or HOBt/EDC hydrochloride (Ito M. et al, 2019, “Heterocylic compound”, WO2019027054), or via aclychloride (Shchekotikhin et al Rus. J. Org. Chem. 2007, 43(11), 1686-1695). All the synthetic routes are summarized in Scheme 1.

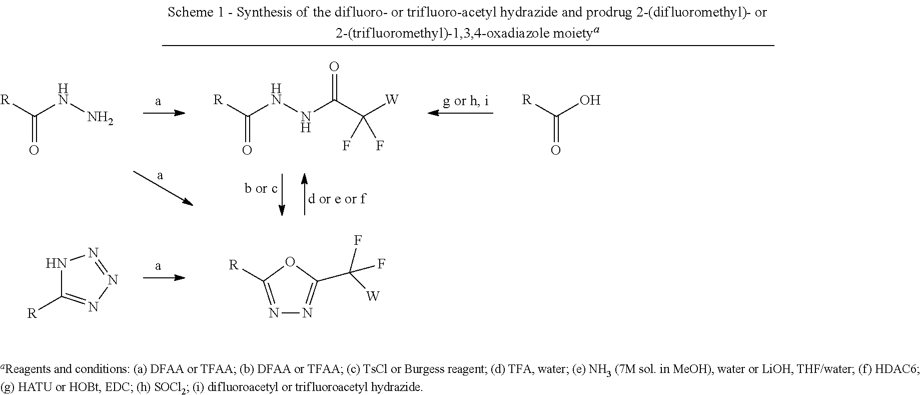

The acylhydrazides object of the present invention were obtained by degradation of the corresponding 2-(difluoromethyl)- or 2-(trifluoromethyl)-1,3,4-oxadiazoles. The synthesis of the parent compounds was obtained as described in the literature, unless otherwise stated.

1,2,3-triazole-based compounds not described elsewhere relied on 2-(4-(bromomethyl)aryl)-5-(difluoromethyl)-1,3,4-oxadiazole or 2-(4-(bromomethyl)aryl)-5-(trifluoromethyl)-1,3,4-oxadiazole common intermediates, whose preparation is described (Marchini, M. et al, 2021, “2-(4-((5-(benzo[b]thiophen-3-yl)-1H-tetrazol-1-yl)methyl)phenyl)-5-(difluoromethyl)-1,3,4-oxadiazole derivatives and similar compounds as selective inhibitors of histone deacetylase 6 (HDAC6) for use in treating e.g. peripheral neuropathy”, WO2022029041). Methyl or ethyl esters were treated with hydrazine to obtain the corresponding hydrazides, which were converted to difluoromethyl- and trifluoromethyl-1,3,4-oxadiazole moieties as described above. Bromomethyl intermediates were then obtained by bromination in benzylic position with N-bromosuccinimide and azobisisobutyronitrile (AIBN) or dibenzoyl peroxide (BPO) as a catalyst (Scheme 2).

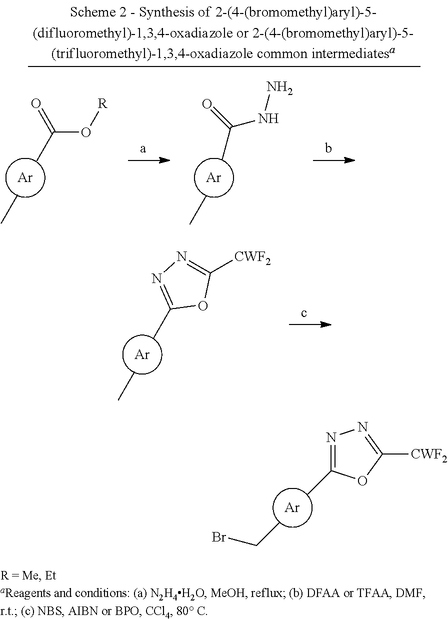

Bromide conversion to azide in the presence of sodium azide and one-pot CuAAC click reaction with an appropriate alkyne gave 1,2,3-triazole containing products bearing 2-(difluoromethyl)- or 2-(trifluoromethyl)-1,3,4-oxadiazole.

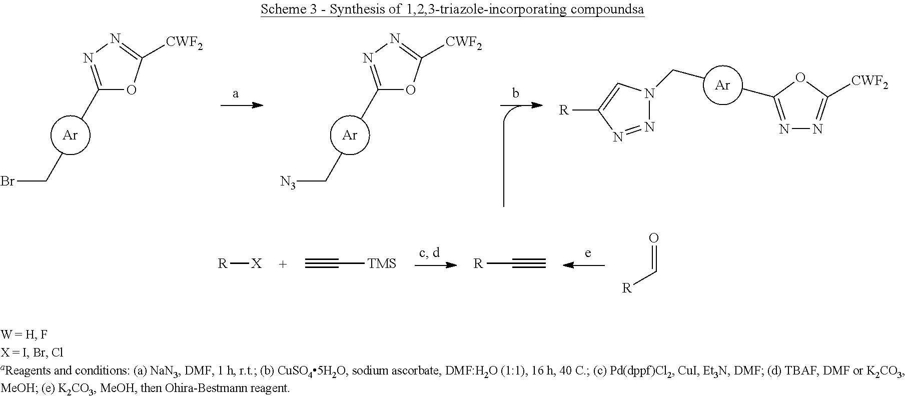

Non-commercial acrylic alkynes were prepared by Sonogashira coupling, reacting a suitable aryl halide with ethynyl(trimethyl)silane in the presence of triethylamine, using [1,1′-Bis(diphenylphosphino)ferrocene]dichloropalladium(II) (Pd(dppf)Cl2) and copper(I) iodide as catalysts (A. G. Sams et al Bioorg. Med. Chem. Lett. 2011, 21(11), 3407-3410), and subsequent cleavage of TMS protection with tetrabutylammonium fluoride (TBAF) or potassium carbonate in methanol. The synthesis of aliphatic alkynes was carried out starting from the corresponding aldehydes under Ohira-Bestmann conditions, with potassium carbonate in methanol (Hönig, M., Carreira, E. M. Angew. Chem. Int. Ed. 2020, 59(3), 1192-1196).

Compounds bearing tetrazole as central scaffolds were synthesized by nucleophilic substitution, reacting the common intermediate 2-(4-(bromomethyl)aryl)-5-(difluoromethyl)-1,3,4-oxadiazole with appropriate substituted tetrazoles at room temperature overnight, in DMF using potassium carbonate as base (see scheme 4).

The reaction provides a mixture of regioisomeric products, which can be efficiently separated by chromatographic methods. 2,5-disubstituted tetrazoles are commonly the most abundant of the two regioisomers. The common intermediate methylbromide-derivative was synthesized as described for 1,2,3-triazole core bearing compounds (scheme 2).

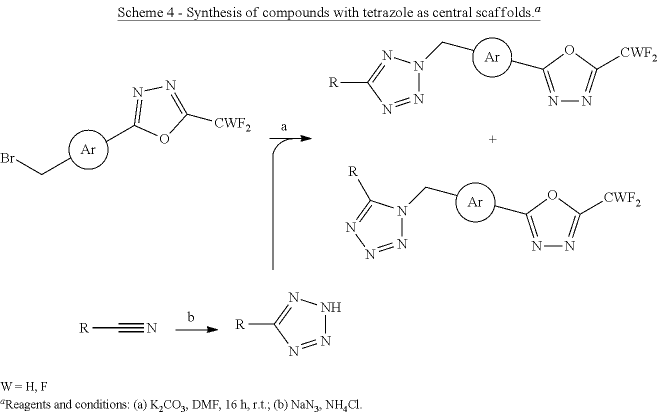

Most of the substituted tetrazoles used were commercially available. Non-commercial building blocks were synthesized from the corresponding carbonitrile by reaction with an excess of sodium azide in the presence of ammonium chloride.

The following examples are intended to further illustrate the invention but not limiting it.

### Example 1. Synthesis of 2-(4-(bromomethyl)-3-chlorophenyl)-5-(difluoromethyl)-1,3,4-oxadiazole (Intermediate C)

### Step 1

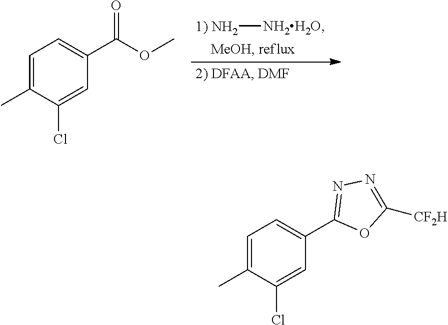

Methyl 3-chloro-4-methylbenzoate (10 g, 54.1 mmol, 1 equiv.) was dissolved in MeOH, and hydrazine hydrate (5 equiv.) was added. The mixture was refluxed under stirring over 5 h, following the conversion to hydrazide by UPLC. The desired intermediate precipitated. It was then collected by filtration and washed with fresh MeOH. The crude residue was dissolved in DMF, and the mixture was cooled to 0° C. DFAA (2.5 equiv.) was added dropwise. The resulting mixture was stirred at r.t. overnight, and full conversion was observed. The reaction mixture was diluted with sat. aq. NaHCO₃. Product precipitated as a solid, which was collected by filtration, rinsed with water and dried under vacuum (8.47 g, 34.6 mmol, 64% yield).

### Step 2

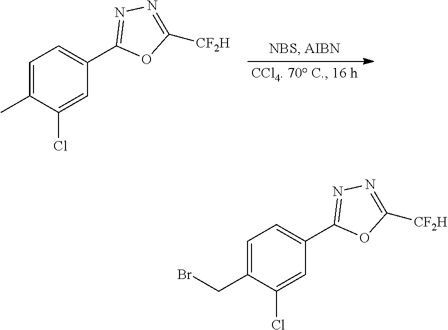 [SMILES: Cc1ccc(-c2nnc(C(F)F)o2)cc1Cl ; FC(F)c1nnc(-c2ccc(CBr)c(Cl)c2)o1]

A mixture of 2-(3-chloro-4-methylphenyl)-5-(difluoromethyl)-1,3,4-oxadiazole (3.44 g, 14 mmol, 1 equiv.) and N-bromosuccinimide (1.1 equiv.) in 70 mL carbon tetrachloride was stirred under argon until complete dissolution. Then AIBN (0.015 equiv.) was added to the reaction mixture, which was stirred at 70° C. overnight. Conversion was followed by LCMS. The mixture was allowed to reach r.t., diluted with DCM and washed successively with sat. aq. NaHCO₃, water and brine. The organic layer was separated, dried over MgSO₄, filtered, and concentrated under reduced pressure. The residue was purified by flash chromatography (silica gel, hexane/EtOAc, 0-15%) affording the desired product as a white solid (2.9 g, 8.9 mmol, 63% yield).

The following compound was prepared according to the same procedure:

| Compound | Structure |
|---|---|
| Intermediate D | 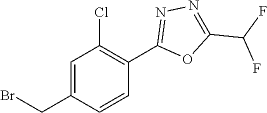 [SMILES: FC(F)c1nnc(-c2ccc(CBr)cc2Cl)o1] |
| | 2-(4-(bromomethyl)-2-chlorophenyl)-5-(difluoromethyl)- |
| | 1,3,4-oxadiazole |

The synthesis of the following analogous intermediates is described elsewhere (Marchini, M. et al, 2022, WO2022029041, example 1):

| Compound | Structure | Reference |
|---|---|---|
| Inter- mediate E | 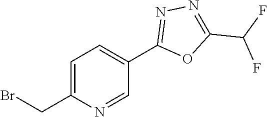 [SMILES: FC(F)c1nnc(-c2ccc(CBr)nc2)o1] | Inter- mediate A |
| | 2-(6-(bromomethyl)pyridin-3-yl)-5- | |
| | (difluoromethyl)-1,3,4-oxadiazole | |
| Inter- mediate F | 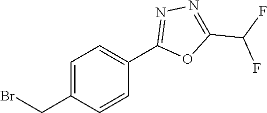 [SMILES: FC(F)c1nnc(-c2ccc(CBr)cc2)o1] | Inter- mediate B |
| | 2-(4-(bromomethyl)phenyl)-5-(difluoromethyl)- | |
| | 1,3,4-oxadiazole | |
| Inter- mediate G | 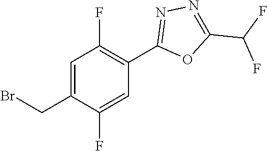 [SMILES: Fc1cc(-c2nnc(C(F)F)o2)c(F)cc1CBr] | Inter- mediate C |
| | 2-(4-(bromomethyl)-3,5-difluorophenyl)-5- | |
| | (difluoromethyl)-1,3,4-oxadiazole | |
| Inter- mediate H | 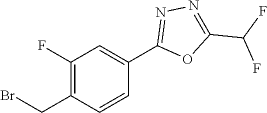 [SMILES: Fc1cc(-c2nnc(C(F)F)o2)ccc1CBr] | Inter- mediate D |
| | 2-(4-(bromomethyl)-3-fluorophenyl)-5- | |
| | (difluoromethyl)-1,3,4-oxadiazole | |
| Inter- mediate I | 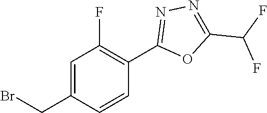 [SMILES: Fc1cc(CBr)ccc1-c1nnc(C(F)F)o1] | Inter- mediate E |
| | 2-(4-(bromomethyl)-2-fluorophenyl)-5- | |
| | (difluoromethyl)-1,3,4-oxadiazole | |
| Inter- mediate J | 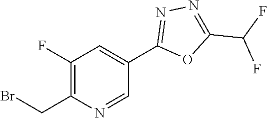 [SMILES: Fc1cc(-c2nnc(C(F)F)o2)cnc1CBr] | Inter- mediate Z |
| | 2-(6-(bromomethyl)-5-fluoropyridin-3-yl)-5- | |
| | (difluoromethyl)-1,3,4-oxadiazole | |

### Example 2. General procedure A for the conversion of 2-(difluoromethyl)- or 2-(trifluoromethyl)-1,3,4-oxadiazole into difluoro or trifluoroacetyl hydrazides

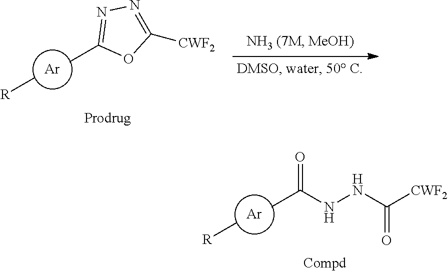

Ammonia (7M solution in MeOH, 10 equiv.) was added to a solution of 2-(difluoromethyl)- or 2-(trifluoromethyl)-1,3,4-oxadiazole bearing parent compound (0.16 mmol, 1 equiv., 0.2M) in DMSO. Water (excess, 1.5-3 mL/mmol) is then added to the resulting mixture, which is heated to 30-70° C. and stirred for 2 days, to achieve full conversion. Ammonia and water are removed under vacuum and the residue is purified by prep HPLC.

The following compounds were prepared following General Procedure A. The synthesis of the parent compound was described elsewhere, as indicated:

| | | [M + H]⁺ | |
|---|---|---|---|
| Compd | Structure | Found | ¹H NMR |
| 13 | 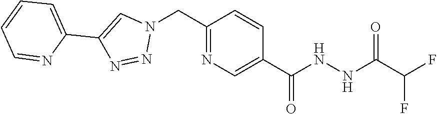 [SMILES: O=C(NNC(=O)C(F)F)c1ccc(Cn2cc(-c3ccccn3)nn2)nc1] | 373.08 | 1H NMR (400 MHz, DMSO-d6) δ 11.02 (s, 2H), 8.98 (d, J = 2.2 Hz, 1H), 8.69 (s, 1H), 8.26 (dd, J = 8.1, 2.3 Hz, 1H), 7.91 − 7.85 (m, 2H), 7.49 − 7.43 (m, 3H), 7.35 (t, J = 7.4 Hz, 1H), 6.39 (t, J = 53.3 Hz, 1H), 5.87 (s, 2H). |
| 14 | 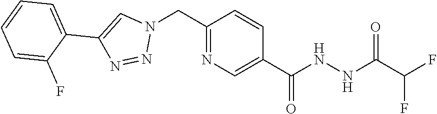 [SMILES: O=C(NNC(=O)C(F)F)c1ccc(Cn2cc(-c3ccccc3F)nn2)nc1] | 391.06 | 1H NMR (400 MHz, DMSO-d6) δ 11.00 (br s, 2H), 8.98 (d, J = 2.2 Hz, 1H), 8.60 (d, J = 3.8 Hz, 1H), 8.25 (dd, J = 8.2, 2.3 Hz, 1H), 8.17 (m, J = 7.6, 1.7 Hz, 1H), 7.47 − 7.42 (m, 2H), 7.39 − 7.32 (m, 3H), 6.42 (t, J = 53.1 Hz, 1H), 5.93 (s, 2H). |
| 39 | 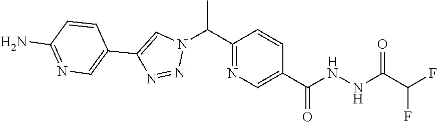 [SMILES: CC(c1ccc(C(=O)NNC(=O)C(F)F)cn1)n1cc(-c2ccc(N)nc2)nn1] | 403.19 | 1H NMR (400 MHz, DMSO-d6) δ 10.36 (s, 2H), 8.97 (d, J = 2.2 Hz, 1H), 8.58 (s, 1H), 8.42 (d, J = 2.3 Hz, 1H), 8.22 (dd, J = 8.2, 2.3 Hz, 1H), 7.83 (dd, J = 8.5, 2.4 Hz, 1H), 7.41 (d, J = 8.2 Hz, 1H), 6.51 (dd, J = 8.6, 0.8 Hz, 1H), 6.28 (t, J = 53.8 Hz, 1H), 6.14 − 6.09 (m, 1H), 6.10 (s, 2H), 1.95 (d, J = 7.1 Hz, 3H). |

| Compd | Prodrug | Parent Structure/Reference |
|---|---|---|
| 13 | 13-A | 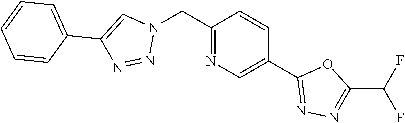 [SMILES: FC(F)c1nnc(-c2ccc(Cn3cc(-c4ccccc4)nn3)nc2)o1] |
| | | WO2022013728, example 16 |
| | | WO2020212479, example 41 |
| 14 | 14-A | 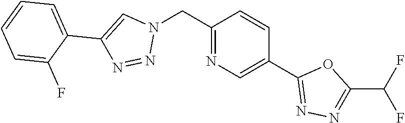 [SMILES: Fc1ccccc1-c1cn(Cc2ccc(-c3nnc(C(F)F)o3)cn2)nn1] |
| | | WO2022013728, example 58 |
| | | WO2020212479, example 48 |
| 39 | 39-A | 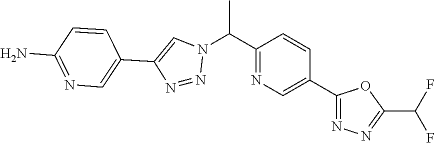 [SMILES: CC(c1ccc(-c2nnc(C(F)F)o2)cn1)n1cc(-c2ccc(N)nc2)nn1] |
| | | WO2022029041, compd 32 |

### Example 3. General procedure B for the conversion of 2-(difluoromethyl)- or 2-(trifluoromethyl)-1,3,4-oxadiazole into difluoro or trifluoroacetyl hydrazides

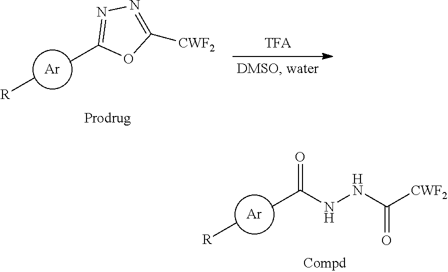

2-(difluoromethyl)- or 2-(trifluoromethyl)-1,3,4-oxadiazole bearing compound (1 equiv., 0.07M) is dissolved in DMSO/water 95:5. TFA (60 equiv.) is added to the mixture, which is stirred at r.t. over 3 days, to achieve full conversion. The reaction mixture is concentrated and the residue is purified by prep HPLC.

The following compound was prepared following General Procedure B. The synthesis of the parent compound was described elsewhere, as indicated:

| | | [M + H]⁺ | |
|---|---|---|---|
| Compd | Structure | Found | ¹H NMR |
| 1 | 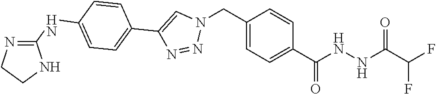 [SMILES: O=C(NNC(=O)C(F)F)c1ccc(Cn2cc(-c3ccc(NC4=NCCN4)cc3)nn2)cc1] | 455.23 | 1H NMR (400 MHz, DMSO-d6) δ 10.19 (s, 4H), 8.64 (s, 1H), 8.36 (s, 1H), 7.88 (d, J = 8.1 Hz, 2H), 7.84 (d, J = 8.6 Hz, 2H), 7.46 (d, J = 8.4 Hz, 2H), 7.24 (d, J = 8.6 Hz, 2H), 6.36 (t, J = 53.4 Hz, 1H), 5.74 (s, 2H), 3.59 (s, 4H). |
| 49 | 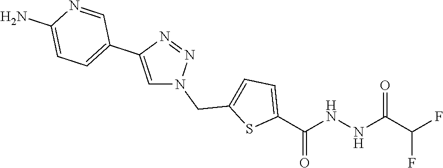 [SMILES: Nc1ccc(-c2cn(Cc3ccc(C(=O)NNC(=O)C(F)F)s3)nn2)cn1] | 394.17 | 1H NMR (300 MHz, DMSO-d6) δ 8.45 (s, 1H), 8.38 (d, J = 2.3 Hz, 1H), 7.80 (dd, J = 8.6, 2.4 Hz, 1H), 7.68 (d, J = 3.8 Hz, 1H), 7.22 (d, J = 3.8 Hz, 1H), 6.49 (s, 1H), 6.32 (t, J = 53.5 Hz, 1H), 6.09 (s, 2H), 5.86 (s, 2H). |

| Compd | Prodrug | Parent Structure/Reference |
|---|---|---|
| 1 | 1-A | 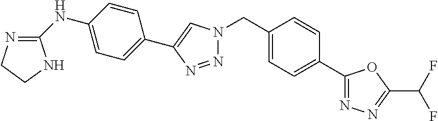 [SMILES: FC(F)c1nnc(-c2ccc(Cn3cc(-c4ccc(NC5=NCCN5)cc4)nn3)cc2)o1] |
| 49 | 49-A | 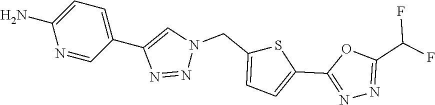 [SMILES: Nc1ccc(-c2cn(Cc3ccc(-c4nnc(C(F)F)o4)s3)nn2)cn1] |

### Example 4. General Procedure C for CuAAC Click Reaction and One-Pot Conversion of 2-(difluoromethyl)- or 2-(trifluoromethyl)-1,3,4-oxadiazole into difluoro or trifluoroacetyl hydrazides

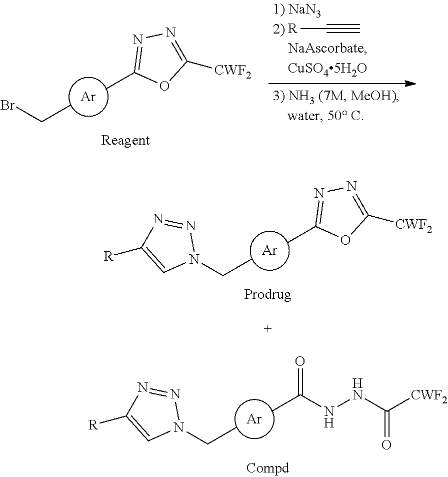

Reagent A (Intermediate A-J example 1, 0.26 mmol, 1 equiv.) was dissolved in 1 mL of DMSO. Sodium azide (0.26 mmol, 1 equiv.) was added and the reaction mixture was stirred for 20 min at r.t. The alkyne (0.26 mmol, 1 equiv.) and solutions of copper(II) sulfate (1M in water, 0.2 equiv.) and (+)-sodium L-ascorbate (0.5M in water, 0.4 equiv.) were added sequentially and the reaction mixture was stirred at r.t. to achieve full consumption of the starting reagents as monitored by UPLC or/and TLC (2-12 hours). 0.5 mL of 7M ammonia solution in MeOH and 0.5 mL of water were added and the reaction mixture was stirred at 50° C. overnight. The reaction mixture was concentrated to remove volatile solvents and the residue was purified by prepHPLC. In some cases, also prodrug could be isolated.

The following compounds were prepared following General Procedure C. The synthesis of alkyne building-block, when not commercially available, was described elsewhere, as indicated:

| | | | [M + H]⁺ | |
|---|---|---|---|---|
| Compd | Structure | Reagent | Found | ¹H NMR |
| 2 | 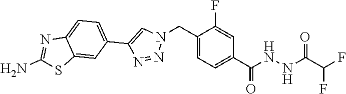 [SMILES: Nc1nc2ccc(-c3cn(Cc4ccc(C(=O)NNC(=O)C(F)F)cc4F)nn3)cc2s1] | Inter. H | 462.18 | 1H NMR (300 MHz, DMSO-d6) δ 10.58 (s, 2H), 8.54 (s, 1H), 8.15 (d, J = 1.7 Hz, 1H), 7.80 − 7.67 (m, 3H), 7.57 (s, 2H), 7.51 (t, J = 7.8 Hz, 1H), 7.37 (d, J = 8.3 Hz, 1H), 6.43 (t, J = 53.0 Hz, 1H), 5.78 (s, 2H). |
| 8 | 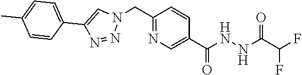 [SMILES: Cc1ccc(-c2cn(Cc3ccc(C(=O)NNC(=O)C(F)F)cn3)nn2)cc1] | Inter. E | 387.11 | 1H NMR (400 MHz, DMSO-d6) δ 10.15 (br s, 2H), 8.98 (d, J = 2.2 Hz, 1H), 8.65 (s, 1H), 8.25 (d, J = 8.1, 2.3 Hz, 1H), 7.71 (s, 1H), 7.66 (d, J = 7.7 Hz, 1H), 7.44 (d, J = 8.2 Hz, 1H), 7.34 (t, J = 7.6 Hz, 1H), 7.16 (d, J = 7.5 |
| | | | | Hz, 1H), 6.35 (t, J = 53.5 Hz, 1H), 5.85 (s, |
| | | | | 2H), 2.37 (s, 3H). |
| 9 | 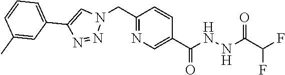 [SMILES: Cc1cccc(-c2cn(Cc3ccc(C(=O)NNC(=O)C(F)F)cn3)nn2)c1] | Inter. E | 387.14 | 1H NMR (400 MHz, DMSO-d6) δ 10.97 (s, 2H), 9.01 − 8.98 (m, 1H), 8.63 (s, 1H), 8.27 (dd, J = 8.1, 2.3 Hz, 1H), 7.79 − 7.74 (m, 2H), 7.47 (d, J = 8.1 Hz, 1H), 7.27 (d, J = 7.9 Hz, 2H), 6.45 (t, J = 53.0 Hz, 1H), 5.86 (s, 2H), |
| | | | | 2.34 (s, 3H). |
| 12 | 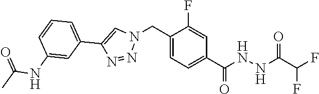 [SMILES: CC(=O)Nc1cccc(-c2cn(Cc3ccc(C(=O)NNC(=O)C(F)F)cc3F)nn2)c1] | Inter. H | 447.20 | 1H NMR (400 MHz, DMSO-d6) δ 10.92 (br s, 2H), 10.04 (s, 1H), 8.60 (s, 1H), 8.13 (t, J = 1.9 Hz, 1H), 7.77 − 7.70 (m, 2H), 7.59 − 7.55 (m, 1H), 7.52 − 7.46 (m, 2H), 7.35 (t, J = 7.9 Hz, 1H), 6.39 (t, J = 53.3 Hz, 1H), 5.79 (s, 2H), 2.06 (s, 3H). |
| 15 | 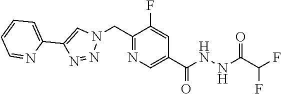 [SMILES: O=C(NNC(=O)C(F)F)c1cnc(Cn2cc(-c3ccccn3)nn2)c(F)c1] | Inter. | 392.27 | 1H NMR (400 MHz, DMSO-d6) o 11.05 (br s, 2H), 8.82 (s, 1H), 8.69 (s, 1H), 8.63 − 8.60 (m, 1H), 8.19 (dd, J = 10.1, 1.7 Hz, 1H), 8.05 (d, J = 7.9 Hz, 1H), 7.91 (td, J = 7.7, 1.8 Hz, 1H), 7.36 (t, J = 6.0 Hz, 1H), 6.46 (t, J = 53.0 Hz, 1H), 6.00 (d, J = 1.8 Hz, 2H). |
| 18 | 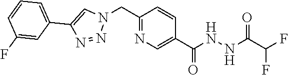 [SMILES: O=C(NNC(=O)C(F)F)c1ccc(Cn2cc(-c3cccc(F)c3)nn2)nc1] | Inter. E | 391.09\| | 1H NMR (400 MHz, DMSO-d6) δ 10.43 (s, 2H), 8.98 (d, J = 2.2 Hz, 1H), 8.77 (s, 1H), 8.31 (s, 1H), 8.26 (dd, J = 8.2, 2.3 Hz, 1H), 7.78 − 7.66 (m, 2H), 7.55 − 7.45 (m, 2H), 7.24 − 7.13 (m, 1H), 6.36 (t, J = 53.5 Hz, 1H), 5.87 (s, 2H). |
| 19 | 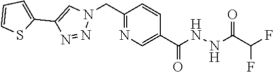 [SMILES: O=C(NNC(=O)C(F)F)c1ccc(Cn2cc(-c3cccs3)nn2)nc1] | Inter. E | 379.36 | 1H NMR (400 MHz, DMSO-d6) o 10.98 (s, 2H), 8.99 (d, J = 2.2 Hz, 1H), 8.60 (s, 1H), 8.27 (dd, J = 8.1, 2.3 Hz, 1H), 7.58 − 7.45 (m, 3H), 7.14 (dd, J = 5.1, 3.5 Hz, 1H), 6.46 (t, J = 52.9 Hz, 1H), 5.87 (s, 2H). |
| 20 | 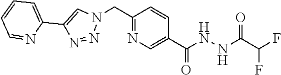 [SMILES: O=C(NNC(=O)C(F)F)c1ccc(Cn2cc(-c3ccccn3)nn2)nc1] | Inter. E | 374.20 | 1H NMR (400 MHz, DMSO-d6) δ 10.25 (br s, 2H), 8.97 (d, J = 2.2 Hz, 1H), 8.73 (s, 1H), 8.65 − 8.59 (m, 1H), 8.33 − 8.22 (m, 2H), 8.08 − 8.04 (m, 1H), 7.95 − 7.88 (m, 1H), 7.45 (d, J = 8.0 Hz, 1H), 7.38 − 7.34 (m, 1H), |
| | | | | 6.38 (t, J = 53.4 Hz, 1H), 5.91 (s, 2H). |
| 27 | 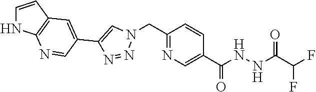 [SMILES: O=C(NNC(=O)C(F)F)c1ccc(Cn2cc(-c3cnc4[nH]ccc4c3)nn2)nc1] | Inter. E | 413.18 | 1H NMR (400 MHz, DMSO-d6) δ 11.74 (s, 1H), 10.82 (br s, 2H), 9.00 (d, J = 2.3 Hz, 1H), 8.74 (d, J = 2.0 Hz, 1H), 8.70 (s, 1H), 8.41 (d, J = 2.0 Hz, 1H), 8.30 (br s, 1H), 8.27 (dd, J = 8.1, 2.3 Hz, 1H), 7.53 − 7.51 (m, 1H), 7.48 (d, J = 8.2 Hz, 1H), 6.51 (dd, J = 3.4, |
| | | | | 1.8 Hz, 1H), 6.37 (t, J = 53.5 Hz, 1H), 5.88 (s, |
| | | | | 2H). |
| 31 | 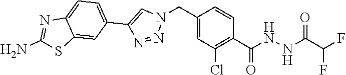 [SMILES: Nc1nc2ccc(-c3cn(Cc4ccc(C(=O)NNC(=O)C(F)F)c(Cl)c4)nn3)cc2s1] | Inter. D | 478.10 | 7.70 (dd, J = 8.3, 1.8 Hz, 1H), 7.57 (s, 3H), 1H NMR (400 MHz, DMSO-d6) δ 8.58 (s, 1H), 8.32 (s, 1H), 8.14 (d, J = 1.7 Hz, 1H), 7.53 (d, J = 7.9 Hz, 1H), 7.41 − 7.33 (m, 2H), 6.26 (t, J = 53.8 Hz, 1H), 5.71 (s, 2H). |
| 32 | 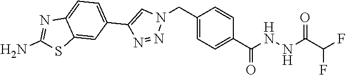 [SMILES: Nc1nc2ccc(-c3cn(Cc4ccc(C(=O)NNC(=O)C(F)F)cc4)nn3)cc2s1] | Inter. F | 444.23 | 1H NMR (400 MHz, DMSO-d6) δ 10.69 (br s, 2H), 8.56 (s, 1H), 8.14 (d, J = 1.8 Hz, 1H), 7.94 − 7.87 (m, 2H), 7.70 (dd, J = 8.3, 1.8 Hz, 1H), 7.57 (s, 2H), 7.47 (d, J = 8.3 Hz, 2H), 7.38 (d, J = 8.3 Hz, 1H), 6.43 (t, J = |
| | | | | 53.0 Hz, 1H), 5.74 (s, 2H). |
| 34 | 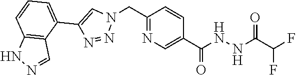 [SMILES: O=C(NNC(=O)C(F)F)c1ccc(Cn2cc(-c3cccc4[nH]ncc34)nn2)nc1] | Inter. E | 413.23 | 1H NMR (400 MHz, DMSO-d6) δ 13.24 (s, 1H), 10.82 (br s, 2H), 9.00 (d, J = 2.2 Hz, 1H), 8.95 (s, 1H), 8.59 (s, 1H), 8.28 (dd, J = 8.2, 2.3 Hz, 1H), 7.65 (d, J = 7.0 Hz, 1H), 7.53 (dd, J = 12.4, 8.2 Hz, 2H), 7.43 (dd, J = |
| | | | | 8.4, 7.1 Hz, 1H), 6.45 (t, J = 53.0 Hz, 1H), |
| | | | | 5.93 (s, 2H). |
| 36 | 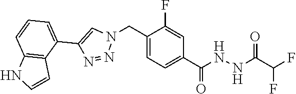 [SMILES: O=C(NNC(=O)C(F)F)c1ccc(Cn2cc(-c3cccc4[nH]ccc34)nn2)c(F)c1] | Inter. H | 429.39 | 1H NMR (400 MHz, DMSO-d6) δ 11.29 (s, 1H), 10.40 (br s, 2H), 8.76 (d, J = 2.2 Hz, 1H), 8.38 (s, 1H), 7.72 (t, J = 8.2 Hz, 2H), 7.57 (d, J = 7.3 Hz, 1H), 7.51 − 7.44 (m, 2H), 7.41 (d, J = 8.1 Hz, 1H), 7.16 (t, J = 7.7 Hz, 1H), 6.96 (t, J = 2.5 Hz, 1H), 6.31 (t, J = 53.7 |
| | | | | Hz, 1H), 5.82 (s, 2H). |
| 37 | 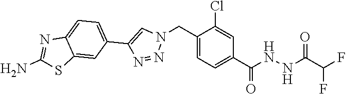 [SMILES: Nc1nc2ccc(-c3cn(Cc4ccc(C(=O)NNC(=O)C(F)F)cc4Cl)nn3)cc2s1] | Inter. C | 478.33 | 1H NMR (400 MHz, DMSO-d6) δ 8.52 (s, 1H), 8.37 (br s, 1H), 8.15 (s, 1H), 7.97 (s, 1H), 7.84 (d, J = 8.0 Hz, 1H), 7.72 (d, J = 9.2 Hz, 1H), 7.57 (s, 2H), 7.37 (dd, J = 8.3, 4.4 Hz, 2H), 6.26 (t, J = 54.2 Hz, 1H), 5.80 (s, 2H). |
| 38 | 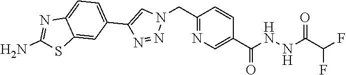 [SMILES: Nc1nc2ccc(-c3cn(Cc4ccc(C(=O)NNC(=O)C(F)F)cn4)nn3)cc2s1] | Inter. E | 445.15 | 1H NMR (400 MHz, DMSO-d6) δ 10.66 (br s, 2H), 8.99 (d, J = 2.2 Hz, 1H), 8.58 (s, 1H), 8.26 (dd, J = 8.2, 2.3 Hz, 1H), 8.16 (d, J = 1.8 Hz, 1H), 7.72 (dd, J = 8.3, 1.8 Hz, 1H), 7.57 (s, 2H), 7.45 (d, J = 8.2 Hz, 1H), 7.38 |
| | | | | (d, J = 8.3 Hz, 1H), 6.38 (t, J = 53.4 Hz, 1H), |
| | | | | 5.85 (s, 2H). |
| 40 | 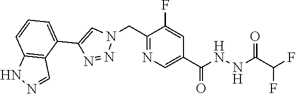 [SMILES: O=C(NNC(=O)C(F)F)c1cnc(Cn2cc(-c3cccc4[nH]ncc34)nn2)c(F)c1] | Inter. J | 431.26 | 1H NMR (400 MHz, DMSO-d6) δ 13.24 (s, 1H), 8.90 (s, 1H), 8.83 (d, J = 1.5 Hz, 1H), 8.58 (s, 1H), 8.29 (s, 1H), 8.19 − 8.12 (m, 1H), 7.64 (d, J = 7.1 Hz, 1H), 7.54 (d, J = 8.3 Hz, 1H), 7.42 (dd, J = 8.4, 7.1 Hz, 1H), 6.33 (t, J = 53.9 Hz, 1H), 5.96 (d, J = 1.9 Hz, 2H). |

| Compd | Prodrug | Parent Structure/Reference |
|---|---|---|
| 2 | 2-A | 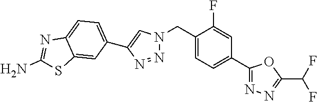 [SMILES: Nc1nc2ccc(-c3cn(Cc4ccc(-c5nnc(C(F)F)o5)cc4F)nn3)cc2s1] |
| | | WO2022029041, compd 339 |
| 8 | 8-A | 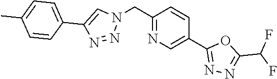 [SMILES: Cc1ccc(-c2cn(Cc3ccc(-c4nnc(C(F)F)o4)cn3)nn2)cc1] |
| | | WO2022013728, example 18 |
| 9 | 9-A | 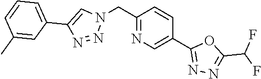 [SMILES: Cc1cccc(-c2cn(Cc3ccc(-c4nnc(C(F)F)o4)cn3)nn2)c1] |
| | | WO2022013728, example 61 |
| 12 | 12-A | 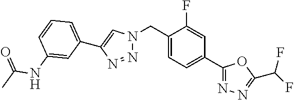 [SMILES: CC(=O)Nc1cccc(-c2cn(Cc3ccc(-c4nnc(C(F)F)o4)cc3F)nn2)c1] |
| | | WO2022029041, as example 26 |
| 15 | 15-A | 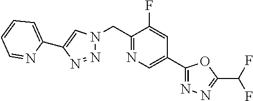 [SMILES: Fc1cc(-c2nnc(C(F)F)o2)cnc1Cn1cc(-c2ccccn2)nn1] |
| | | WO2022013728, example 521 |
| 18 | 18-A | 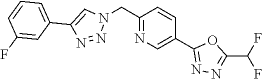 [SMILES: Fc1cccc(-c2cn(Cc3ccc(-c4nnc(C(F)F)o4)cn3)nn2)c1] |
| | | WO2022013728, example 59 |
| 19 | 19-A | 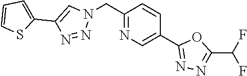 [SMILES: FC(F)c1nnc(-c2ccc(Cn3cc(-c4cccs4)nn3)nc2)o1] |
| | | WO2022013728, example 83 |
| 20 | 20-A | 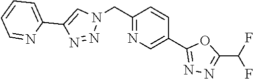 [SMILES: FC(F)c1nnc(-c2ccc(Cn3cc(-c4ccccn4)nn3)nc2)o1] |
| | | WO2022013728, example 68 |
| 27 | 27-A | 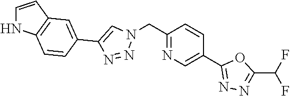 [SMILES: FC(F)c1nnc(-c2ccc(Cn3cc(-c4ccc5[nH]ccc5c4)nn3)nc2)o1] |
| | | WO2022013728, example 52 |
| 31 | 31-A | 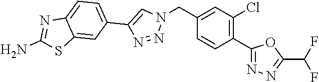 [SMILES: Nc1nc2ccc(-c3cn(Cc4ccc(-c5nnc(C(F)F)o5)c(Cl)c4)nn3)cc2s1] |
| | | WO2022029041, as example 34 |
| 32 | 32-A | 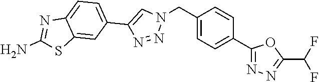 [SMILES: Nc1nc2ccc(-c3cn(Cc4ccc(-c5nnc(C(F)F)o5)cc4)nn3)cc2s1] |
| | | WO2022029041, compd 14 |
| 34 | 34-A | 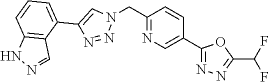 [SMILES: FC(F)c1nnc(-c2ccc(Cn3cc(-c4cccc5[nH]ncc45)nn3)nc2)o1] |
| | | WO2022013728, example 248 |
| 36 | 36-A | 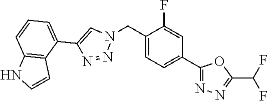 [SMILES: Fc1cc(-c2nnc(C(F)F)o2)ccc1Cn1cc(-c2cccc3[nH]ccc23)nn1] |
| | | WO2022013728, example 157 |
| 37 | 37-A | 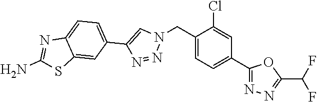 [SMILES: Nc1nc2ccc(-c3cn(Cc4ccc(-c5nnc(C(F)F)o5)cc4Cl)nn3)cc2s1] |
| | | WO2022029041, compd 340 |
| 38 | 38-A | 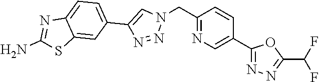 [SMILES: Nc1nc2ccc(-c3cn(Cc4ccc(-c5nnc(C(F)F)o5)cn4)nn3)cc2s1] |
| | | WO2022029041, compd 1 |
| 40 | 40-A | 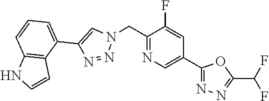 [SMILES: Fc1cc(-c2nnc(C(F)F)o2)cnc1Cn1cc(-c2cccc3[nH]ccc23)nn1] |
| | | WO2022013728, example 579 |

### Example 5. General Procedure D for Tetrazole Nucleophilic Substitution and One-Pot Conversion of 2-(difluoromethyl)- or 2-(trifluoromethyl)-1,3,4-oxadiazole into difluoro or trifluoroacetyl hydrazides

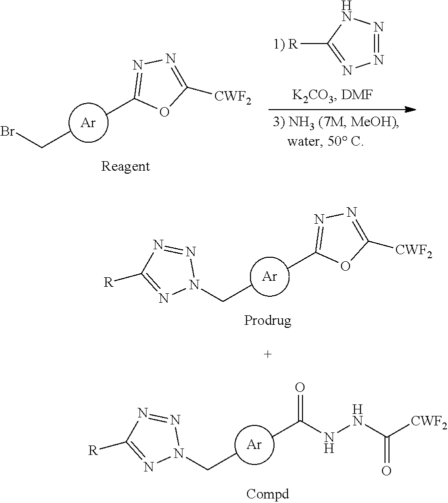

Tetrazole (0.34 mmol, 1 equiv.) was dissolved in 2 mL DMF. The suitable Reagent (Intermediate A-J, example 1, 1 equiv.) and potassium carbonate (2 equiv.) were added. After stirring the reaction mixture for 3 h (or until full conversion), methanolic ammonia (7M, 5 equiv.) and excess of water were added. The resulting mixture was stirred at 50° C. overnight, concentrated and submitted for prepHPLC.

The following compounds were prepared following General Procedure D. The synthesis of prodrug was also described elsewhere, as indicated:

| | | | [M − | |
|---|---|---|---|---|
| | | | H]⁻ | |
| Compd | Structure | Reagent | Found | ¹H NMR |
| 16 | 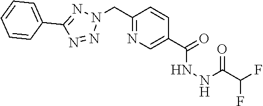 [SMILES: O=C(NNC(=O)C(F)F)c1ccc(Cn2nnc(-c3ccccc3)n2)nc1] | Inter. E | 372.05 | 1H NMR (400 MHz, DMSO-d6) δ 10.93 (s, 2H), 8.99 − 8.94 (m, 1H), 8.30 (dd, J = 8.1, 2.3 Hz, 1H), 8.11 − 8.03 (m, 2H), 7.63 (d, J = 8.5 Hz, 1H), 7.57 (dd, J = 5.2, 2.0 Hz, 3H), 6.45 (t, J = 52.9 Hz, 1H), 6.25 (s, 2H). |
| 17 | 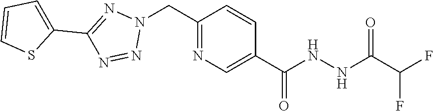 [SMILES: O=C(NNC(=O)C(F)F)c1ccc(Cn2nnc(-c3cccs3)n2)nc1] | Inter. E | 378.01 | 1H NMR (400 MHz, DMSO-d6) δ 10.98 (s, 2H), 8.95 (dd, J = 2.3, 0.9 Hz, 1H), 8.29 (dd, J = 8.1, 2.3 Hz, 1H), 7.85 − 7.77 (m, 2H), 7.66 − 7.59 (m, 1H), 7.26 (dd, J = 5.0, 3.6 Hz, 1H), 6.45 (t, J = 52.9 Hz, 1H), 6.23 (s, 2H). |

| Compd | Prodrug | Parent Structure/Reference |
|---|---|---|
| 16 | 16-A | 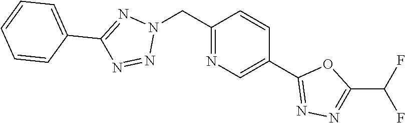 [SMILES: FC(F)c1nnc(-c2ccc(Cn3nnc(-c4ccccc4)n3)nc2)o1] |
| | | WO2022013728, example 195 |
| 17 | 17-A | 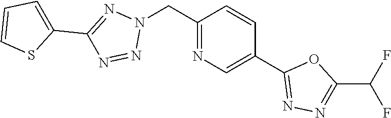 [SMILES: FC(F)c1nnc(-c2ccc(Cn3nnc(-c4cccs4)n3)nc2)o1] |
| | | WO2022013728, example 193 |

### Example 6. Synthesis of 4-(1-(4-(2-aminobenzo[d]thiazol-6-yl)-1H-1,2,3-triazol-1-yl)ethyl)-N′-(2,2-difluoroacetyl)-3-fluorobenzohydrazide (Compd. 30) and 6-(1-(1-(4-(5-(difluoromethyl)-1,3,4-oxadiazol-2-yl)-2-fluorophenyl)ethyl)-1H-1,2,3-triazol-4-yl)benzo[d]thiazol-2-amine (Compd. 30-A)

### Step A

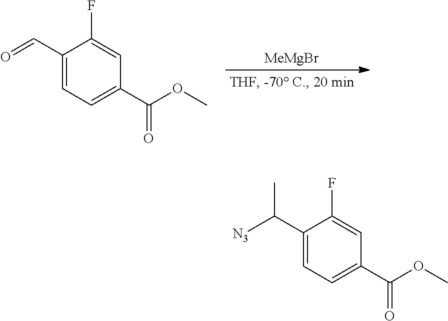 [SMILES: "MeMgBr" ; COC(=O)c1ccc(C=O)c(F)c1 ; COC(=O)c1ccc(C(C)N=[N+]=[N-])c(F)c1]

A solution of methyl methyl 3-fluoro-4-formylbenzoate (1 g, 5.49 mmol, 1 equiv.) in THF (20 mL) was cooled down to −70° C. Methylmagnesium bromide (1 equiv.) was added dropwise, and the resulting mixture was stirred for 20 min at −70° C. The reaction was then quenched with NH₄Cl aq. sol. and extracted with MTBE. Organic layers were merged, dried over Na₂SO₄, filtered, and concentrated. Crude was purified by flash chromatography (silica gel, hexane/EtOAc 0-30%) to obtain the desired product (1.09 g; 5.49 mmol, 100% yield).

### Step B

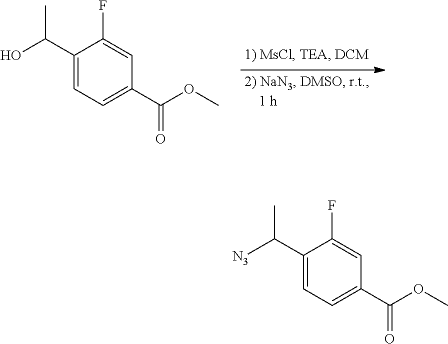 [SMILES: COC(=O)c1ccc(C(C)O)c(F)c1 ; COC(=O)c1ccc(C(C)N=[N+]=[N-])c(F)c1]

Methyl 3-fluoro-4-(1-hydroxyethyl)benzoate (1.09 g; 5.49 mmol, 1 equiv.) was dissolved in 20 mL DCM. Triethylamine (2 equiv.) and methanesulfonyl chloride (1.2 equiv.) were added, and the mixture was stirred at r.t. overnight. Full conversion was observed. Reaction mixture was diluted with EtOAc, washed with brine, dried over Na₂SO₄, filtered, and concentrated.

Crude intermediate mesylate was dissolved in 10 mL DMSO and sodium azide (1 equiv.) was added. The resulting mixture was stirred at r.t. over 45 min, then diluted with MTBE, and washed with brine. Organic layer was dried over Na₂SO₄, filtered, and concentrated. The product thus obtained was used in the next step without further purification (1.2 g, 5.38 mmol, 98% yield).

### Step C

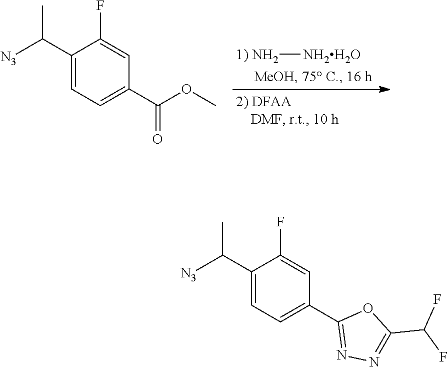

Hydrazide monohydrate (6 equiv.) was added to a solution of methyl 4-(1-azidoethyl)-3-fluorobenzoate (1.2 g, 5.38 mmol, 1 equiv.) in 10 mL MeOH. The reaction mixture was refluxed under stirring for 3 h, and then evaporated to dryness.

Intermediate hydrazide was dissolved in 5 mL DMF, and difluoroacetic anhydride (2.5 equiv.) was added. The reaction mixture was stirred at r.t. overnight, diluted with sat. aq. NaHCO₃, and extracted with MTBE. Organic layer was dried over Na₂SO₄, filtered, and concentrated. Crude was purified by flash chromatography (silica gel, hexane/EtOAc 0-15%) to obtain the desired product (860 mg, 3.04 mmol, 56% yield).

### Step D

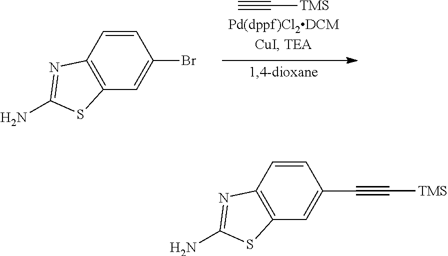

6-bromo-1,3-benzothiazol-2-amine (8 g, 34.9 mmol, 1 equiv.) was dissolved in 75 mL dioxane. Triethylamine (2 equiv.) was added, and the mixture was degassed with Ar. Copper iodide (0.1 equiv.) and [1,1′-Bis(diphenylphosphino)ferrocene]dichloropalladium(II) DCM complex (0.1 equiv.) were added and the mixture was degassed again. Ethynyl(trimethyl)silane (3 equiv.) was added, and the mixture was stirred at 95° C. overnight. The reaction mixture was let to reach r.t., then it was diluted with EtOAc, and filtered over celite. Filtrate solution was washed with 5% NH₃ aq. solution, then with sat. aq. NaHCO₃ and brine. Organic phase was then dried over Na₂SO₄, filtered, and concentrated to dryness. Crude was purified by flash chromatography (silica gel, 20-50% Hex/EtOAc), to obtain 7.38 g of the desired intermediate (29.9 mmol, 86% yield).

### Step E

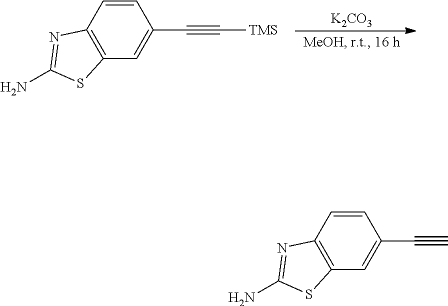

6-((trimethylsilyl)ethynyl)benzo[d]thiazol-2-amine (7.38 g, 29.9 mmol, 1 equiv.) was suspended in 75 mL MeOH and potassium carbonate (1.5 equiv.) was added. The resulting mixture was stirred at r.t. overnight to obtain full conversion. Crude was purified by flash chromatography (silica gel, dry-load, 0-4% MeOH/DCM) to obtain 4.2 g of the desired intermediate (24.1 mmol, 80% yield).

### Step F-1

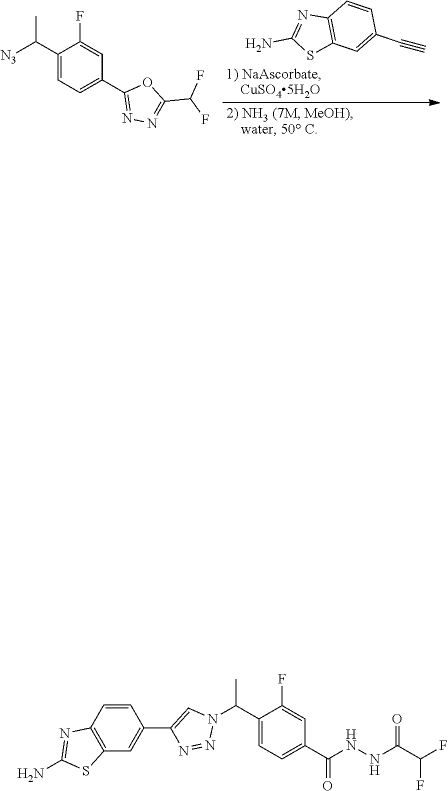

2-[4-(1-azidoethyl)-3-fluorophenyl]-5-(difluoromethyl)-1,3,4-oxadiazole (60 mg, 0.34 mmol, 1 equiv.) and 6-ethynyl-1,3-benzothiazol-2-amine (60 mg, 0.34 mmol, 1 equiv.) were solubilized in 2 mL DMSO. Copper sulfate pentahydrate (0.3 equiv., 0.5M aq. sol.) and (+)-sodium L-ascorbate (0.5 equiv., 1M aq. sol.) were added and the mixture was stirred at r.t. overnight. UPLC showed full conversion to the desired click reaction product.

The reaction mixture was heated to 50° C. A mixture of water (0.5 mL) and methanolic ammonia (0.5 mL, 7M sol.) was added to the reaction mixture, which was stirred at 50° C. overnight. Full conversion to the desired product was detected by UPLC. The mixture was concentrated by rotary evaporation, filtered, and submitted for purification. RP-flash chromatography and successive prep-HPLC gave 12.3 mg of the desired product as a free base (0.02 mmol, 7% yield).

Compd. 30: [M+H]⁺ found 476.35; ¹H NMR (400 MHz, DMSO-d6) δ 10.82 (br s, 2H), 8.69 (s, 1H), 8.15 (d, J=1.7 Hz, 1H), 7.80-7.69 (m, 3H), 7.57 (s, 2H), 7.52 (t, J=7.8 Hz, 1H), 7.38 (d, J=8.3 Hz, 1H), 6.45 (t, J=52.9 Hz, 1H), 6.26 (q, J=7.0 Hz, 1H), 1.97 (d, J=7.0 Hz, 3H).

### Step F-2

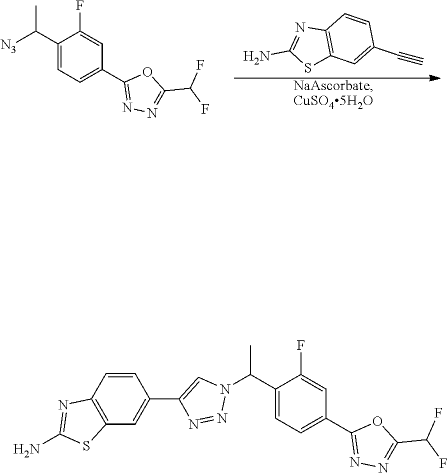

2-[4-(1-azidoethyl)-3-fluorophenyl]-5-(difluoromethyl)-1,3,4-oxadiazole (400 mg, 1.4 mmol, 1 equiv.) and 6-ethynyl-1,3-benzothiazol-2-amine (246 mg, 1.4 mmol, 1 equiv.) were dissolved in 5 mL DMSO. Copper sulfate pentahydrate (0.1 equiv., 0.5M aq. sol.) and (+)-sodium L-ascorbate (0.2 equiv., 1M aq. sol.) were added and the mixture was stirred at r.t. over 1 h. UPLC showed full conversion to the desired product. The mixture was submitted directly for RP-flash chromatography (water/ACN 40%, 0.1% FA) to obtain pure product (566 mg, 1.24 mmol, 88% yield).

Compd. 30-A: [M+H]⁺ found 458.17; ¹H NMR (400 MHz, DMSO-d6) δ 8.73 (s, 1H), 8.32 (s, 1H), 8.15 (d, J=1.7 Hz, 1H), 7.97-7.91 (m, 2H), 7.75-7.68 (m, 1H), 7.63-7.55 (m, 4H), 7.38 (d, J=8.3 Hz, 1H), 6.30 (q, J=7.0 Hz, 1H), 1.99 (d, J=7.1 Hz, 3H).

### Example 7. Synthesis of 5-[[4-(2-amino-1,3-benzothiazol-6-yl)triazol-1-yl]methyl]-N′-(2,2-difluoroacetyl)thiophene-2-carbohydrazide (comp. 48)

### Step 1

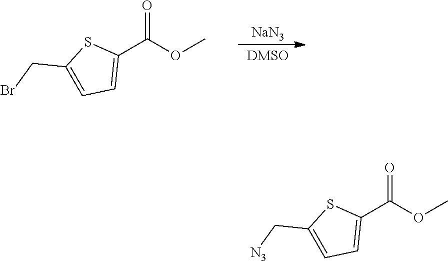

methyl 5-(bromomethyl)thiophene-2-carboxylate (1 g, 4.2 mmol, 1 equiv.) was dissolved in 8 mL DMSO and sodium azide (1.05 equiv.) was added. After 1 h full conversion was observed by LCMS. Water was added and the reaction mixture was extracted with EtOAc and concentrated under reduced pressure. Crude product was used in the next step without purification.

### Step 2

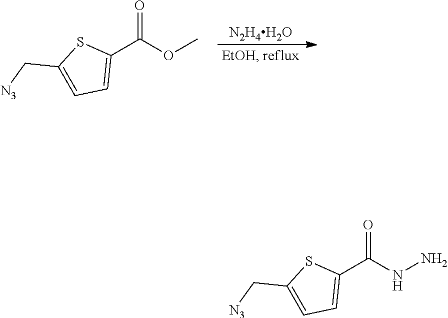

methyl 5-(azidomethyl)thiophene-2-carboxylate (402 mg, 2 mmol, 1 equiv.) was dissolved in 10 mL ethanol and hydrazine (5 equiv.) was added. The reaction mixture was refluxed under stirring overnight, and then concentrated under reduced pressure. The crude residue thus obtained was used in the next step without purification.

### Step 3

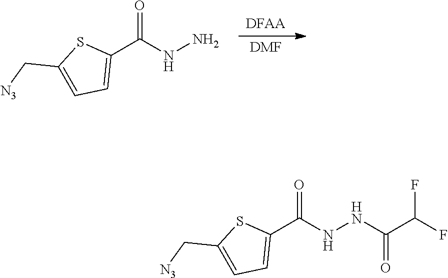

Difluoroacetic anhydride (1 equiv.) was added dropwise to a solution of 5-(azidomethyl)thiophene-2-carbohydrazide (200 mg, 1.0 mmol, 1 equiv.) in 5 mL DMF. After 1 h water was added and the reaction mixture was extracted with EtOAc, dried over Na₂SO₄ and concentrated under reduced pressure. The product obtained (210 mg) was used in the next step without any further purification.

### Step 4

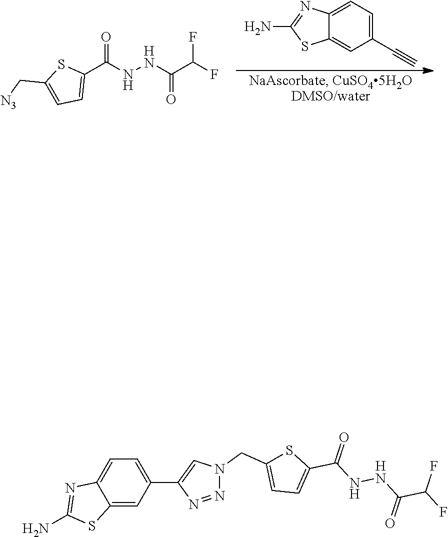 [SMILES: [N-]=[N+]=NCc1ccc(C(=O)NNC(=O)C(F)F)s1 ; C#Cc1ccc2nc(N)sc2c1 ; "NaAscorbate, CuSO4·5H2ODMSO/water" ; Nc1nc2ccc(-c3cn(Cc4ccc(C(=O)NNC(=O)C(F)F)s4)nn3)cc2s1]

5-(azidomethyl)-N′-(2,2-difluoroacetyl)thiophene-2-carbohydrazide (210 mg, 0.51 mmol, 1 equiv.) and 6-ethynyl-1,3-benzothiazol-2-amine (1 equiv.) were dissolved in 3 mL DMSO. Copper sulfate pentahydrate (0.2 equiv.) and sodium ascorbate (0.4 equiv.) were added as aqueous solutions. After 2 h the reaction mixture was filtered and purified by prepHPLC, to obtain the final product (120 mg, 0.26 mmol, 52% yield). [M+H]⁺ found 450.23; ¹H NMR (400 MHz, DMSO) δ 10.98 (s, 1H), 10.70 (s, 1H), 8.56 (s, 1H), 8.15 (d, J=1.7 Hz, 1H), 7.75 (d, J=3.8 Hz, 1H), 7.71 (dd, J=8.3, 1.8 Hz, 1H), 7.57 (s, 2H), 7.38 (d, J=8.3 Hz, 1H), 7.27 (d, J=3.8 Hz, 1H), 6.44 (t, J=52.9 Hz, 1H), 5.91 (s, 2H).

### Example 8. General Procedure E for the Bio-Conversion of 2-(difluoromethyl)- or 2-(trifluoromethyl)-1,3,4-oxadiazole into difluoro or trifluoroacetyl hydrazides

HDAC6 (1 μM) was incubated at 25° C. with 5 μM prodrug compound in assay buffer. At different times, aliquots (40 μL) were transferred to test tubes containing acetonitrile (240 μL) to quench the reaction. Samples were kept frozen at −80° C. until LC-HRMS analysis.

The LC-HRMS analysis was carried out using a Vanquish Flex UHPLC (Thermo Fisher Scientific) and a high-resolution mass spectrometer Orbitrap QExactive Focus (Thermo Fisher Scientific), equipped with a Heated Electrospray Ionization, operated in positive mode. A Full Scan analysis was set in the m/z range 50-500 amu. A XSelect HSS T3 50×2.1 mm, 2.5 μm chromatographic column (Waters) was used. Mobile phase A consisted of 0.1% formic acid in water and mobile phase B in 0.1% formic acid in acetonitrile. The flow rate was set to 0.5 mL/min, with a gradient program from 3 to 20% B in 3 minutes.

Disappearance of prodrug was observed as function of time when incubated with HDAC6 enzyme. HR-LCMS analysis showed the formation of a new compound having molecular weight=MW[_{prodrug}]+18, corresponding to acylhydrazide.

The following compounds were obtained and identified using general procedure E:

| Compd | Structure | [M + H]⁺ Found | Prodrug |
|---|---|---|---|
| 45 | 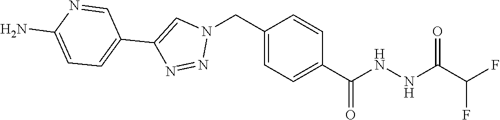 [SMILES: Nc1ccc(-c2cn(Cc3ccc(C(=O)NNC(=O)C(F)F)cc3)nn2)cn1] | 388,1328 | 45-A |
| 46 | 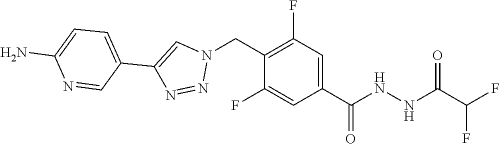 [SMILES: Nc1ccc(-c2cn(Cc3c(F)cc(C(=O)NNC(=O)C(F)F)cc3F)nn2)cn1] | 424,1140 | 46-A |
| 47 | 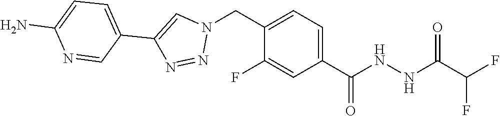 [SMILES: Nc1ccc(-c2cn(Cc3ccc(C(=O)NNC(=O)C(F)F)cc3F)nn2)cn1] | 406,1234 | 47-A |
| 42 | 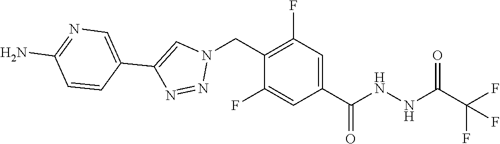 [SMILES: Nc1ccc(-c2cn(Cc3c(F)cc(C(=O)NNC(=O)C(F)(F)F)cc3F)nn2)cn1] | 442,1046 | 42-A |
| 43 | 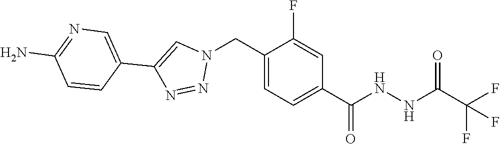 [SMILES: Nc1ccc(-c2cn(Cc3ccc(C(=O)NNC(=O)C(F)(F)F)cc3F)nn2)cn1] | 424,1140 | 43-A |
| 44 | 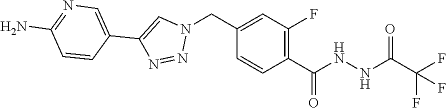 [SMILES: Nc1ccc(-c2cn(Cc3ccc(C(=O)NNC(=O)C(F)(F)F)c(F)c3)nn2)cn1] | 424,1140 | 44-A |

| Compd | Parent Structure / Reference |
|---|---|
| 45 | 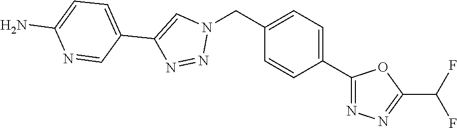 [SMILES: Nc1ccc(-c2cn(Cc3ccc(-c4nnc(C(F)F)o4)cc3)nn2)cn1] |
| | WO2022029041; compd 52 |
| 46 | 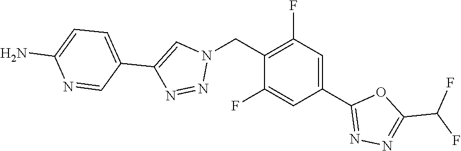 [SMILES: Nc1ccc(-c2cn(Cc3c(F)cc(-c4nnc(C(F)F)o4)cc3F)nn2)cn1] |
| | WO2022029041; compd 42 |
| 47 | 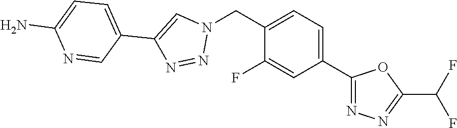 [SMILES: Nc1ccc(-c2cn(Cc3ccc(-c4nnc(C(F)F)o4)cc3F)nn2)cn1] |
| | WO2022029041; compd 48 |
| 42 | 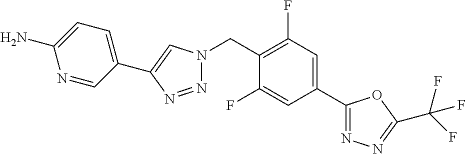 [SMILES: Nc1ccc(-c2cn(Cc3c(F)cc(-c4nnc(C(F)(F)F)o4)cc3F)nn2)cn1] |
| 43 | 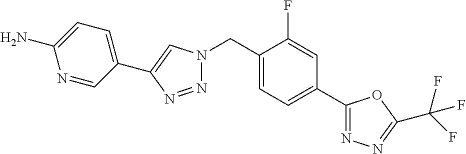 [SMILES: Nc1ccc(-c2cn(Cc3ccc(-c4nnc(C(F)(F)F)o4)cc3F)nn2)cn1] |
| 44 | 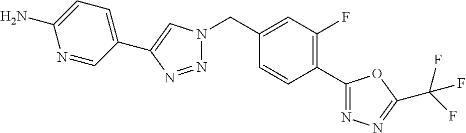 [SMILES: Nc1ccc(-c2cn(Cc3ccc(-c4nnc(C(F)(F)F)o4)c(F)c3)nn2)cn1] |

### Example 9. LC-HRMS Analysis for Monitoring Enzymatic Reaction

HDAC6 (1 μM) were incubated at 25° C. with 5 μM DFMO-compound in assay buffer. At different times, aliquots (40 μL) were transferred to test tubes containing acetonitrile (240 μL) to quench the reaction. Samples were kept frozen at −80° C. until LC-HRMS analysis. Quantitation of test item in the samples was performed with respect to calibration curves obtained with varying concentrations of compounds. The calculated concentrations of compounds were plotted in table 1 as percentage of compounds concentration at each time point.

The LC-HRMS analysis was carried out using a Vanquish Flex UHPLC (Thermo Fisher Scientific) and a high-resolution mass spectrometer Orbitrap QExactive Focus (Thermo Fisher Scientific), equipped with a Heated Electrospray Ionization, operated in positive mode. A Full Scan analysis was set in the m/z range 50-500 amu. A XSelect HSS T3 50×2.1 mm, 2.5 μm chromatographic column (Waters) was used. Mobile phase A consisted of 0.1% formic acid in water and mobile phase B in 0.1% formic acid in acetonitrile. The flow rate was set to 0.5 mL/min, with a gradient program from 3 to 20% B in 3 minutes.

TABLE 1
Kinetic of prodrug disappearing when incubated with HDAC6

| Time | | | | | | | | | |
|---|---|---|---|---|---|---|---|---|---|
| (min) | 31-A | 45-A | 2-A | 37-A | 42-A | 47-A | 43-A | 46-A | 44-A |
| 0 | 100.00 | 100.00 | 100.00 | 100.00 | 100.00 | 100.00 | 100.00 | 100.00 | 100.00 |
| 30 | 89.4 | 79.9 | 73.1 | 88.1 | 61.6 | 53.6 | 56.9 | 42.4 | 19.9 |
| 60 | 76.5 | 73.7 | 53.6 | 69.2 | 39.2 | 29.7 | 27.8 | 17.4 | 0.5 |
| 120 | 56.4 | 47.8 | 31.1 | 38.4 | 12.8 | 7.4 | 2.9 | 2.7 | 0.1 |
| 240 | 28.4 | 22.6 | 11.1 | 12.1 | 0.1 | 0.4 | 0.0 | 0.0 | 0.0 |
| 360 | 15.0 | 11.6 | 3.9 | 3.7 | 0.0 | 0.0 | 0.0 | 0.0 | 0.0 |
| 480 | 7.9 | 5.2 | 1.3 | 1.3 | 0.0 | 0.0 | 0.0 | 0.0 | 0.0 |
| T_{1/2} | 129.2 | 113.4 | 77.5 | 74.3 | 40.5 | 29.3 | 23.1 | 22.9 | 11.3 |
| (min) | | | | | | | | | |

For all tested compounds it was possible to register the disappearance when incubated with HDAC6 enzyme. HR-LCMS analysis also showed the formation of a new compound with molecular weight=MW+18, corresponding to the hydrate form.

For compounds 2-A, 31-A and 37-A, retention time in HPLC analysis confirmed that the forming products were the corresponding compounds 2, 31 and 37.

All compounds were also incubated in the same buffer for the same time in absence of enzyme and they all resulted to be stable (>80% up to 8 hours, except for example 42-A, which was 60% residual after 8 h).

### Example 10. Isolation of Long-Lived/Tight Complex

Rapid chromatography on spin columns coupled to LC-HRMS was used to attempt the isolation of long-lived/tight complexes between HDAC6 and examples 1 and 1-A. zHDAC6-CD2 wt (1 μM) was incubated with 1 (5 μM) or 1-A (also 5 μM) as described before for 6 hours. An aliquot (60 μL) was loaded on a Bio-Spin P-6 Gel Column (Bio-Rad) that had been equilibrated with assay buffer in order to separate the HDAC6-inhibitors complexes from free compounds. After centrifugation to elute the initial fraction containing most of the enzyme, ten 200 μL aliquots were applied and eluted by centrifugation to collect the low molecular weight (free) compounds. Aliquots of the fractions were subjected to LC-HRMS analysis (as described above) for identification and quantitation of 1 and its derivatives.

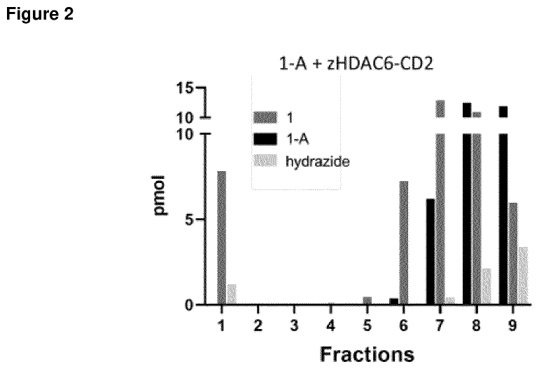

From the graph of FIG. 2 clearly appears that the only compound that co-elutes with the enzyme (fraction 1) is compound 1, suggesting that this is the real inhibitor, while compound 1-A behaves as a prodrug (note that at this extreme concentration also hydrazide, the hydrolysis product of compound 1, is formed and it binds the enzyme, too, even if in a small extent).

### Example 11. Enzymatic Screening

For each test compound, 100× concentrated DMSO solutions at 8 doses were prepared and then diluted in assay buffer (25 mM Tris-HCl, pH 8, 130 mM NaCl, 0.05% Tween-20, 10% Glycerol) to obtain 5× concentrated solutions in relation to the final concentrations (typical final concentration range—6.4-200000 nM or 0.18-50000 nM, final DMSO content—1%). Then 10 μL solution of each test compound concentration were placed on a 96-well plate in triplicate and 15 μL of 3.33× concentrated enzyme solution in the assay buffer containing 3.33× concentrated BSA (final BSA concentration 1 mg/mL) and, in case of HDAC6, 3.33× concentrated TCEP (final TCEP concentration—200 μM) were added to each well. After a period of preincubation at 25° C. (30 minutes for HDAC6 and 120 minutes for HDAC1), 25 μL of solution containing the substrate were added. As substrate, FLUOR DE LYS® deacetylase substrate (Enzo Life Sciences, cat: BML-KI104, FdL), FLUOR DE LYS®-Green substrate (Enzo Life Sciences, cat: BML-KI572, FdL_G) or Boc-Lys(Tfa)-AMC (Bachem, cat: 4060676.005, Tfal)—2× concentrated solution in assay buffer were used. Following a reaction period (30 minutes at 25° C.), 50 μL of the development solution consisting of concentrate FLUOR DE LYS® developer I (Enzo Life Sciences, ca: BML-KI105), diluted 200 times in buffer (50 mM Tris-HCl, pH 8, 137 mM NaCl, 2.7 mM KCl, 1 mM MgCl₂) plus 2 μM TSA was added and, after 25 minutes at room temperature in the dark, using the Victor 1420 Multilabel Counter Perkin Elmer Wallac instrument, the fluorescence reading was carried out (excitation/emission: 485/535 nM—Fluor de Lys Green, 355/460 nM—Tfal, Fluor de Lys).

Enzymatic activity on recombinant human HDAC6 and HDAC1 was evaluated (Table 2) for each synthesized compound.

TABLE 2
Enzyme Inhibitory Activity Assay on
HDAC6 and on HDAC1 (IC₅₀ in nM unit).

| | HDAC6 | HDAC1 |
|---|---|---|
| Compound | IC₅₀, nM | IC₅₀, nM |
| 1-A | 14 | >100000 |
| 2-A | 24 | >100000 |
| 8-A | 65 | >100000 |
| 9-A | 40 | >100000 |
| 12-A | 57 | >100000 |
| 13-A | 32 | >100000 |
| 14-A | 22 | >100000 |
| 15-A | 100 | >100000 |
| 16-A | 40 | >100000 |
| 17-A | 41 | >100000 |
| 18-A | 56 | >100000 |
| 19-A | 31 | >100000 |
| 20-A | 60 | >100000 |
| 27-A | 15 | >100000 |
| 30-A | 67 | >100000 |
| 31-A | 594 | >100000 |
| 32-A | 26 | >100000 |
| 34-A | 20 | >100000 |
| 36-A | 54 | >100000 |
| 37-A | 61 | >100000 |
| 38-A | 8 | >100000 |
| 39-A | 14 | >100000 |
| 40-A | 44 | >100000 |
| 42-A | 183 | >100000 |
| 43-A | 85 | >100000 |
| 44-A | 410 | >100000 |
| 45-A | 26 | >100000 |
| 46-A | 19 | >100000 |
| 47-A | 21 | >100000 |
| 48-A | 7 | >100000 |
| 49-A | 9 | 16217 |

All compounds tested resulted virtually inactive (IC₅₀>100 PM) on HDAC1, while showing relevant activity on HDAC6 (IC₅₀<600 nM). This confirms the selectivity associated with this class of compounds.

### Example 12. In Vitro α-Tubulin Acetylation in 697 Cell Lines

The in vitro α-tubulin acetylation was evaluated on human B cell precursor leukemia 697.

The 697 cells were maintained in RPMI Medium 1640 (Gibco, cat: 21875-034) supplemented with 10 mM HEPES (Gibco, cat: 15630-080), Pen-Strep (Penicillin 100 U/ml, Streptomycin 100 μg/ml, Gibco, cat: 15140-122) and 10% fetal bovine serum (Gibco, cat: 10270-106).

The cells were plated in 12-well plates (Costar, cat: 3512) at the density of 5.5×10⁵ cells/ml.

Serial dilutions of test compounds in DMSO were prepared using 20 mM stock solutions to obtain 8 doses 200× concentrated in respect to final doses (2.7-100000 nM). Then the DMSO solutions were diluted 10× in culture medium to obtain 20× concentrated solutions which were used for cells treatment (125 μl of medium solutions were added to 2.375 ml of cells suspension). The final DMSO content was set as 0.5%. The plates were incubated at 37° C., 5% CO₂ for 16 hours.

At the end of the incubation period, the cells were harvested and centrifuged for 5 minutes at 200×g and washed with 0.9% NaCl at 4° C. The resulting pellet was treated for 30 minutes at 4° C. with 100 μl Complete Lysis-M buffer containing protease inhibitors (Complete Lysis-M Roche+Complete Tablets, Mini Easypack, cat: 4719956001) and phosphatase inhibitor cocktails (PhosStop Easypack, Roche, cat: 4906837001) and then centrifuged 10 minutes at 18213×g. The protein concentration in each supernatant was determined using BCA Protein Assay Kit (Pierce, cat: 23227). The samples were diluted in PBS 1× to obtain 2 μg/ml concentration and coated in MaxiSorp 96-well plates (Nunc, cat: 442404). The plates were incubated overnight at room temperature.

Plates were washed twice with Wash Buffer (PBS 1×+0.005% tween 20) and saturated for 1 hour at room temperature with 300 μL of 1×PBS containing 10% FBS. After washing twice with Wash Buffer, the plates were incubated for 2 hours at room temperature in the presence of 100 μl/well either anti-acetylated-α-tubulin antibody (Monoclonal Anti-Tubulin, Acetylated antibody produced in mouse, Sigma-Aldrich, cat: T6793) or total anti-α-tubulin antibody (Monoclonal Anti-α-Tubulin produced in mouse, Sigma-Aldrich, cat: T6074) diluted 1:1000 in 1×PBS containing 10% FBS. Following 5 washing cycles with Wash Buffer the secondary antibody conjugated with the enzyme HRP (Goat anti-Mouse IgG, IgM, IgA (H+L), stock concentration 0.5 mg/ml, Thermo Fisher Scientific, cat: A10668), diluted 1:1000 in 1×PBS+10% FBS was added at the volume of 100 μl/well. After two hours of incubation at room temperature the plates were washed 4 times with Wash Buffer, then 100 μl/well of TMB substrate (TMB substrate kit, Thermo Fisher Scientific, cat: 34021) was added for 10 minutes at room temperature in the dark. The reaction was stopped by adding 50 μl of 2M H2SO4. The plates were read in BioTek Synergy H1 multimode microplate reader at a wavelength of 450 nm.

The measured absorbance was corrected by subtracting the mean of blank values (samples without the primary antibody). The absorbance ratios of acetyl to total tubulin assays were calculated and normalized to the reference compound (positive control) 4 parameter logistic curve, where 0% is the fitted bottom and 100% is the fitted top of the curve. The results are expressed as relative EC₅₀.

TABLE 3
Tubulin acetylation in 697 cell line (EC₅₀ in
nM unit) for a selected number of compounds.

| | TubAc 697 cells |
|---|---|
| Example | EC50, nM |
| 1-A | 585 |
| 2-A | 130 |
| 8-A | 419 |
| 9-A | 385 |
| 12-A | 346 |
| 13-A | 505 |
| 14-A | 476 |
| 16-A | 1086 |
| 17-A | 648 |
| 18-A | 582 |
| 19-A | 579 |
| 20-A | 1208 |
| 27-A | 119 |
| 30-A | 1391 |
| 32-A | 85 |
| 34-A | 167 |
| 36-A | 167 |
| 37-A | 65 |
| 38-A | 76 |
| 39-A | 320 |
| 40-A | 849 |
| 45-A | 664 |
| 46-A | 788 |
| 47-A | 871 |
| 48-A | 7.5 |
| 49-A | 29 |

All tested compounds show very high activity in inducing tubulin acetylation in 697 cell line.

### Example 13. In Vitro α-Tubulin Acetylation in N2a Cell Lines

The in vitro α-tubulin acetylation was evaluated on murine neuroblastoma N2a cell lines.

Cells were maintained in Eagle's Minimum Essential Medium (ATCC, cat: 30-2003) supplemented with 10% fetal bovine serum—FBS (Gibco, cat: 10270-106).

Cells were plated in 12-well plates (Costar, cat: 3512) at the density of 6×10⁴ cells/cm², respectively. The test compounds were prepared as 20× concentrated medium solutions in respect to the final concentrations. The cells were treated the following day. The compounds were tested at 3 doses: 10 μM, 1 μM and 0.1 μM. The final DMSO content was set as 0.5%. The cells were incubated with the compounds at 37° C. for 16 hours.

At the end of the incubation period, the cells were harvested and centrifuged for 5 minutes at 200×g and washed with 0.9% NaCl at 4° C. The resulting pellet was treated for 30 minutes at 4° C. with 100 μl Complete Lysis-M buffer containing protease inhibitors (Complete Lysis-M Roche+Complete Tablets, Mini Easypack, cat: 4719956001) and phosphatase inhibitor cocktails (PhosStop Easypack, Roche, cat: 4906837001) and then centrifuged 10 minutes at 18213×g. The protein concentration in each supernatant was determined using BCA Protein Assay Kit (Pierce, cat: 23227). The samples were diluted in PBS 1× to obtain 2 μg/ml concentration and coated in MaxiSorp 96-well plates (Nunc, cat: 442404). The plates were incubated overnight at room temperature, then washed twice with Wash Buffer (PBS 1×+0.005% tween 20) and saturated for 1 hour at room temperature with 300 μL of 1×PBS containing 10% FBS. After washing twice with Wash Buffer, the plates were incubated for 2 hours at room temperature in the presence of 100 μl/well either anti-acetylated-α-tubulin antibody (Monoclonal Anti-Tubulin, Acetylated antibody produced in mouse, Sigma-Aldrich, cat: T6793) or total anti-α-tubulin antibody (Monoclonal Anti-α-Tubulin produced in mouse, Sigma-Aldrich, cat: T6074) diluted 1:1000 in 1×PBS containing 10% FBS. Following 5 washing cycles with Wash Buffer, the secondary antibody conjugated with the enzyme HRP (Goat anti-Mouse IgG, IgM, IgA (H+L), stock concentration 0.5 mg/ml, Thermo Fisher Scientific, cat: A10668), diluted 1:1000 in 1×PBS+10% FBS was added at the volume of 100 μl/well. After two hours of incubation at room temperature the plates were washed 4 times with Wash Buffer, then 100 μl/well of TMB substrate (TMB substrate kit, Thermo Fisher Scientific, cat: 34021) was added for 15 minutes at room temperature in the dark. The reaction was stopped by adding 50 μl of 2M H₂SO₄. The plates were read in BioTek Synergy H1 multimode microplate reader at a wavelength of 450 nm.

The measured absorbance was corrected by subtracting the mean of blank values (samples without the primary antibody). The absorbance ratios of acetyl to total tubulin assays were calculated and normalized to the reference compound (positive control) 4 parameter logistic curve, where 100% is the fitted top of the curve and 0% is DMSO control (protein extract obtained from non-treated cells). The results are expressed as fold increase compared to the control (DMSO).

TABLE 4
Tubulin acetylation in N2a cell line (fold increase compared
to control) for a selected number of compounds

| Example | Fold increase |
|---|---|
| 13-A | 6 |
| 14-A | 7 |
| 20-A | 15 |
| 32-A | 20 |
| 37-A | 31 |
| 38-A | 22 |
| 48-A | 45 |
| 49-A | 26 |

All tested compounds show very high activity in inducing tubulin acetylation in N2a cell line.

### Example 14. In Vitro α-Tubulin Acetylation in Undifferentiated SH-SY5Y Cell Lines

SH-SY5Y cells (ATCC, cod. CRL-2266) are plated in optical optimized 96-well black plates (Perkin Elmer, cod. 6055302) at 5000 cells/well in 100 μl/well of growth medium (DMEM/F12 (1:1)+10 mM hepes+100 units/mL of penicillin+100 μg/mL of streptomycin+10% of inactivated Foetal calf serum (FCS, Hyclone)).

After 24 h of seeding, cells are incubated overnight with the selected molecules at 0.1-1-10 μM. ACY1083 and Tubastatin A, at the same doses, are tested as positive controls of α-Tubulin acetylation, while untreated cells are incubated with 0.01% DMSO and indicated as CTRL DMSO.

At the end of the incubation, cells are fixed adding 100 μL/well of formaldehyde 8% in PBS (final formaldehyde concentration 4% in 200 μL/well) directly in the 100 μL/well of medium, for 30 min at RT. The fixing solution is carefully removed, and wells are washed twice with PBS for 10 min each. Fixed cells are kept in PBS at 4° C. until staining.

The day of the staining experiment, the fixed cells are incubated 60 min with the blocking buffer (PBS with 5% FCS+0.3% Triton™ X-100). While blocking, primary antibodies are prepared by diluting 1:200 the α-Tubulin Alexa Fluor 488 Conjugate (Cell Signaling, cod. 5063) antibody and 1:50 Acetyl-α-Tubulin Alexa Fluor 647 Conjugate (Cell Signaling, cod. 81502) antibody in the Antibody Dilution Buffer (PBS with 1% BSA+0.3% Triton™ X-100). Once the blocking solution is aspirated, the diluted primary antibodies are applied and incubated overnight at 4° C. The next day, cells are rinsed twice with PBS (10 min each), incubated 5 min with 300 nM DAPI in PBS, and then rinsed twice with PBS (10 min each). For each treatment, 3 wells are stained.

Images of the stained cells are acquired by means of the IN Cell Analyzer 2500 HS Instrument, using: far red channel for Acetyl-α-Tubulin staining (exposure 0.02 sec), green channel for α-Tubulin staining (exposure 0.02 sec), and blue channel for DAPI (nuclei) staining. For each well, 10 images are acquired.

Images of stained cells are analysed with the InCarta software (Molecular Devices) for obtaining fluorescence intensity considering whole cells. For each treatment, mean values of Cell intensity—Bckg (Cell), for both staining, are obtained using the InCarta raw data by FOV (Field of View). Results are expressed as fold increase of the ratio of acetylated tubulin and total tubulin towards control (CTRL DMSO).

TABLE 5
Tubulin acetylation in indiferentiated SH-SY5Y cell
line (fold increase of the ratio of acetylated tubulin
and total tubulin towards control at 1 μM).

| | TubAc SH-SY5Y cells |
|---|---|
| Example | fold increase @1uM |
| 1-A | 2 |
| 2-A | 6.2 |
| 14-A | 6.3 |
| 32-A | 6.3 |
| 37-A | 6 |
| 38-A | 10 |
| 39-A | 6.5 |
| 48-A | 15.7 |
| 49-A | 13.9 |

All tested compounds show very high activity in inducing tubulin acetylation in SH-SY5Y cell line.

1-10. (canceled)

11. A compound of formula (I), or a pharmaceutically acceptable salt or isomer thereof: 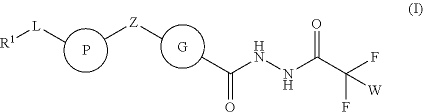
wherein:
W is H or F;
G is a 5-membered heteroaromatic ring consisting of carbon atoms and 1 to 4 heteroatoms selected from N, O, S and Se, which ring is optionally substituted with C₁-C₃ alkyl, alkoxy, or thioalkoxy, halogenated derivatives thereof, or halogen, or hydroxy;
or G is a substructure of formula (Ia): 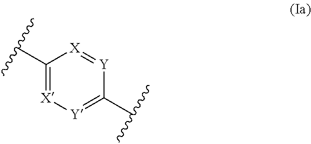
wherein X, X′, Y and Y′ are independently selected from CH, N, CF or CCl;
Z is —CD₂-, —CF₂—, —CHR²—, —NH—, —S—;
R² is H, halogen, C₁-C₆ alkyl or C₃-C₆ cycloalkyl, either unsubstituted or substituted with:
hydroxy, carbonyl, C₁-C₃ alkoxy, aryloxy or thioalkoxy, or halogenated derivatives thereof;
halogen;
primary, secondary, or tertiary amine, substituted with C₁-C₆ alkyl, C₃-C₆ cycloalkyl or halogenated derivatives thereof;
phenyl, pyridyl, thiophenyl, furan or pyrrole, either unsubstituted or substituted with C₁-C₃ alkyl, alkoxy, thioalkoxy or halogenated derivatives thereof, or halogen; or
the following substructures or halogenated derivatives thereof: 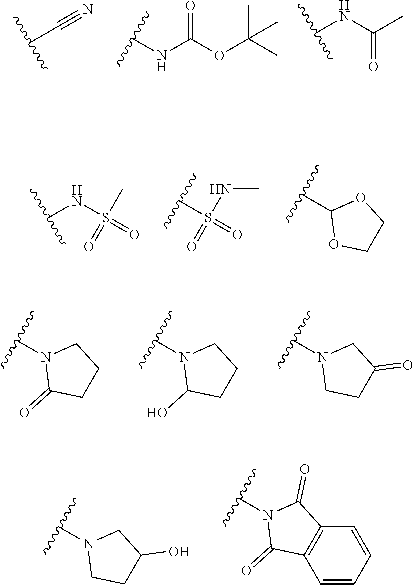
L is absent, C₁-C₆ alkyl, alkoxy or thioalkoxy, —(CH₂)ₘ—CHR⁴—(CH₂)ₒ—, —(CH₂)ₘ—CH(NHR⁴)—(CH₂)ₒ—, —(CH₂)ₘ—NR⁴—(CH₂)ₒ— or halogenated derivatives thereof;
wherein m and o are each independently 0, 1 or 2; or
L is selected from the following substructures (IIa)-(IIf) and halogenated derivatives thereof: 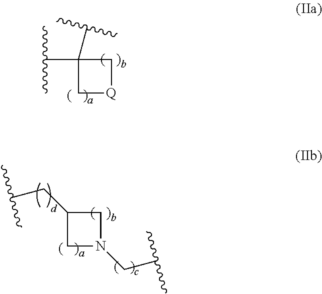
wherein a, b, c and d are independently 0, 1, 2, or 3, and a and b cannot be 0 at the same time;
Q is CH₂, NR⁴ or O; 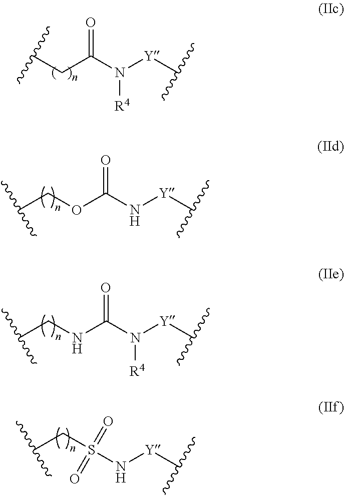
wherein n is 0, 1, or 2;
Y″ is absent, C₁-C₂ alkenyl, or is selected from the following substructures and halogenated derivatives thereof: 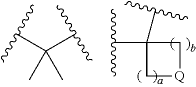
wherein a, b and Q are as defined above;
R⁴ is H, C₁-C₄ alkyl unsubstituted or substituted with:
halogen
phenyl, pyridyl, thiophenyl, furan or pyrrole, either unsubstituted or substituted with C₁-C₃ alkyl, alkoxy, thioalkoxy or halogenated derivatives thereof, or halogen;
P is an unsubstituted or substituted, aromatic or non-aromatic 5 to 10 membered heterocyclic ring, wherein the ring consists of carbon atoms and one or more heteroatoms selected from N, O and S;
R¹ is absent, halogen, unsubstituted or substituted —C(═O)C₁-C₄ alkyl, C₁-C₄ alkyl, C₄-C₆ cycloalkenyl, C₆-C₁₂ aryl, or 5- to 9-membered heteroaryl including at least one heteroatom selected from N, O and S;
with the proviso that the following compounds are excluded:
3-[2-[[4-[[(2,2-difluoroacetyl)amino]carbamoyl]phenyl]methyl]tetrazol-5-yl]benzoic acid,
2-((5-(6-aminopyridin-3-yl)-2H-tetrazol-2-yl)methyl)-N′-(2,2-difluoroacetyl)pyrimidine-5-carbohydrazide,
tert-butyl (5-(1-((6-(2-(2,2-difluoroacetyl)hydrazine-1-carbonyl)pyridazin-3-yl)methyl)-1H-1,2,3-triazol-4-yl)pyridin-2-yl)carbamate,
N′-(2,2-difluoroacetyl)-4-((5,5-dimethyl-2,4-dioxo-3-phenylimidazolidin-1-yl)methyl)-3-fluorobenzohydrazide,
4-((5,5-dimethyl-2,4-dioxo-3-phenylimidazolidin-1-yl)methyl)-3-fluoro-N′-(2,2,2-trifluoroacetyl)benzohydrazide,
N′-(2,2-difluoroacetyl)-4-((2,5-dioxo-3-phenylimidazolidin-1-yl)methyl)-3-fluorobenzohydrazide,
N′-(2,2-difluoroacetyl)-6-((1-(3-fluorophenyl)-8-(oxetan-3-yl)-2,4-dioxo-1,3,8-triazaspiro[4.5]decan-3-yl)methyl)nicotinohydrazide,
4-((1H-imidazol-1-yl)methyl)-N′-(2,2-difluoroacetyl)benzohydrazide,
N′-(2,2-difluoroacetyl)-4-(pyrimidin-2-ylamino)benzohydrazide,
benzyl 4-((5-(2-(2,2-difluoroacetyl)hydrazine-1-carbonyl)pyrimidin-2-yl)amino)-4-phenylpiperidine-1-carboxylate, and
4-((1-(3-chloro-4-cyanophenyl)-3,5-dimethyl-1H-pyrazol-4-yl)methyl)-N′-(2,2,2-trifluoroacetyl)benzohydrazide.

12. The compound according to claim 11, wherein R² is H, or C₁-C₃ alkyl, either unsubstituted or substituted with:
C₁-C₃ alkyl or C₃-C₆ cycloalkyl
hydroxy, carbonyl, C₁-C₂ alkoxy, aryloxy or thioalkoxy, or halogenated derivatives thereof;
halogen;
primary, secondary, or tertiary amine, substituted with C₁-C₃ alkyl, C₃-C₆ cycloalkyl or halogenated derivatives thereof;
phenyl, pyridyl, thiophenyl, furan or pyrrole, either unsubstituted or substituted with C₁-C₃ alkyl, alkoxy, thioalkoxy or halogenated derivatives thereof, or halogen; or
the following substructures or halogenated derivatives thereof:

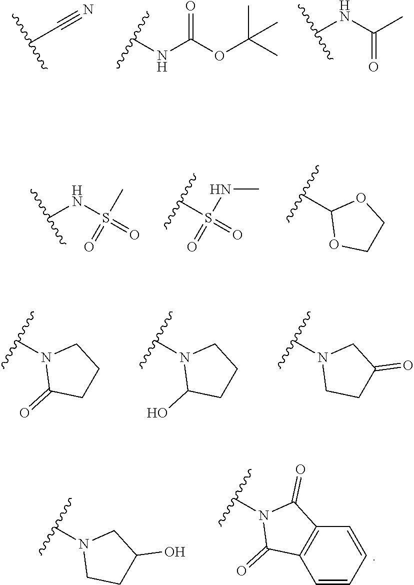

13. The compound according to claim 11, wherein P is 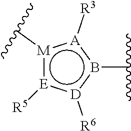
wherein:
A is C, N, O, or S;
B is C or N;
D is C, N, or O;
E is C, N, or O;
M is C or N;
R⁵ and R⁶ are independently —H, halogen, ═O, C₁-C₆ alkyl, alkoxy or thioalkoxy, C₃-C₆ cycloalkyl, or halogenated derivatives thereof, optionally substituted with carbonyl or carboxy, or R⁵ and R⁶ are independently selected from the following substructures: 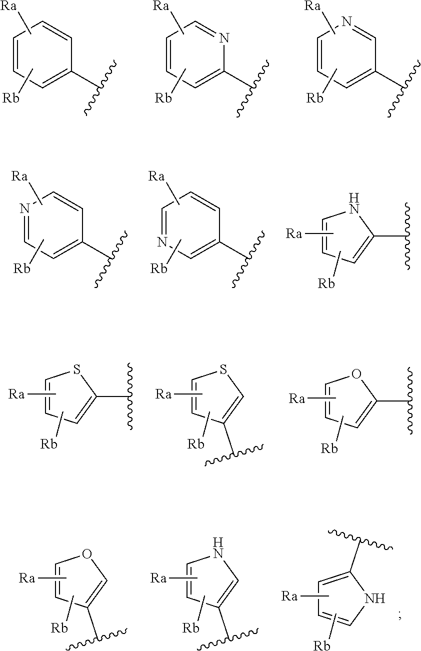
R³ is absent, —H, C₁-C₆ alkyl optionally substituted with —OH or —N(C₁-C₅ alkyl)₂, -LR¹ or is selected from the following substructures: 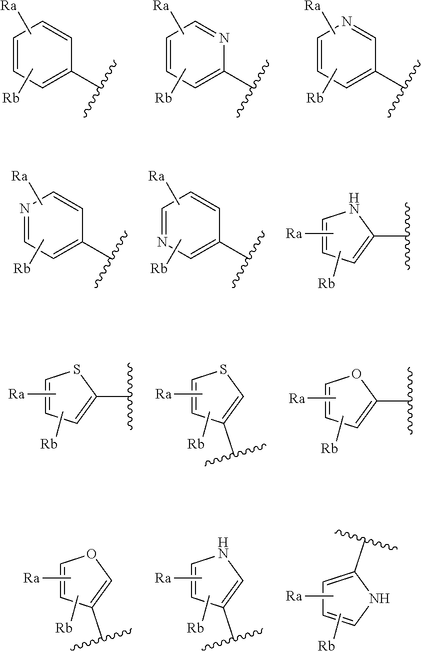
when R³ is -LR¹, substitution on M is absent;
Ra and Rb are independently H, halogen, C₁-C₆ alkyl, alkoxy or thioalkoxy, C₃-C₆ cycloalkyl, or halogenated derivatives thereof.

14. The compound according to claim 11, wherein P is selected from the following substructures: 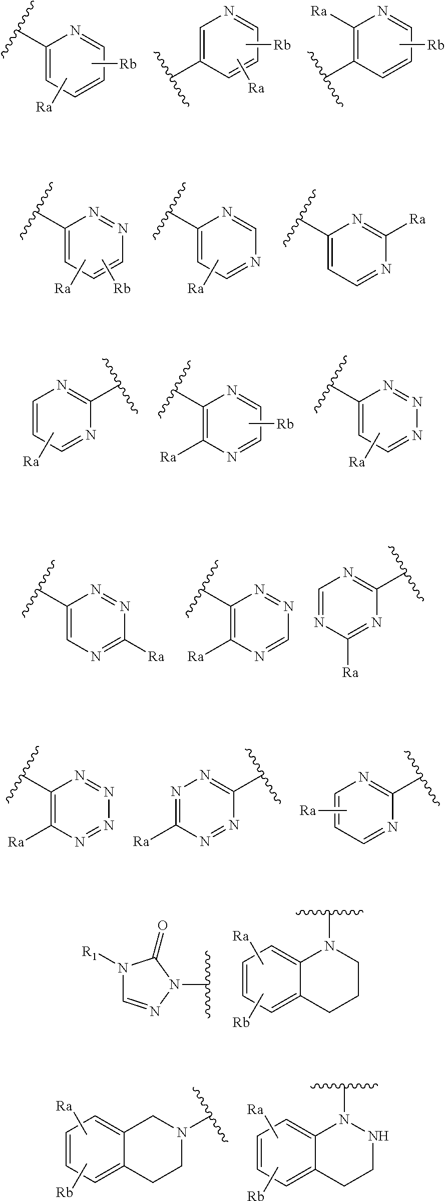 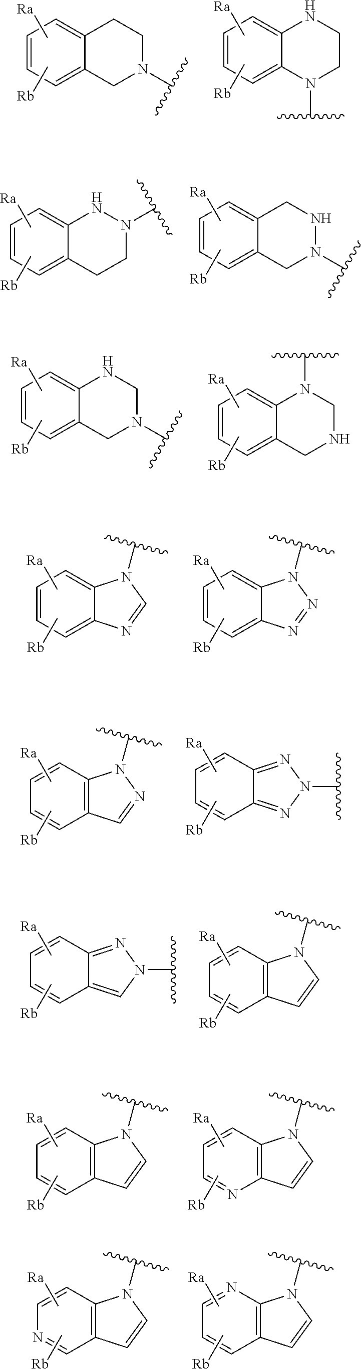 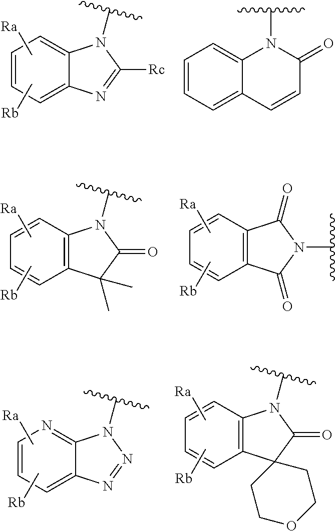
wherein Ra and Rb are independently H, halogen, C₁-C₆ alkyl, alkoxy, thioalkoxy, C₃-C₆ cycloalkyl, or halogenated derivatives thereof, or -L-R¹, wherein L and R¹ are as defined in claim 11;
Rc is H, halogen, C₁-C₆ alkyl, alkoxy or thioalkoxy, C₃-C₆ cycloalkyl, or halogenated derivatives thereof, or —NH₂.

15. The compound according to claim 11, wherein R¹ is selected from the following substructures: 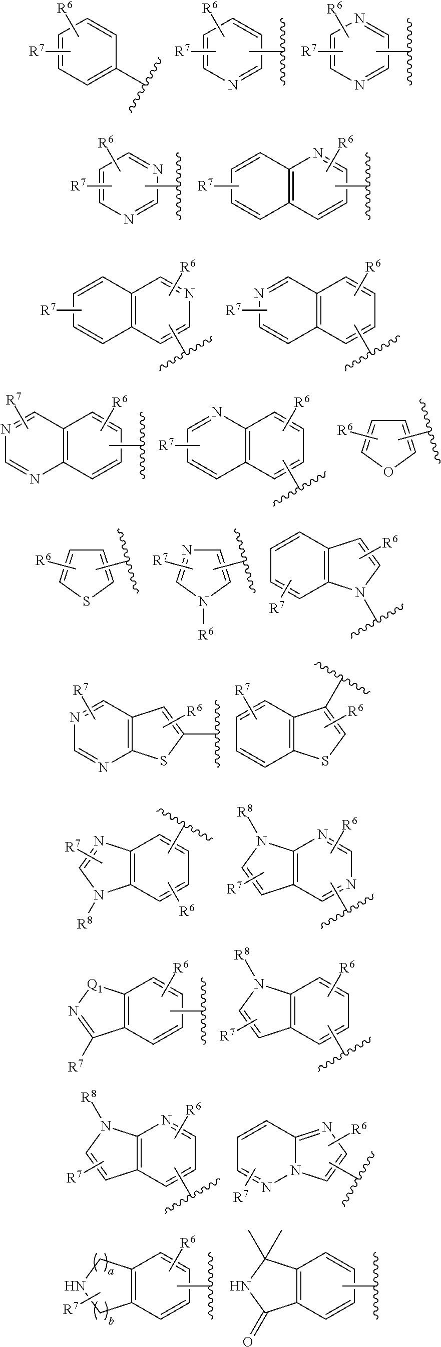 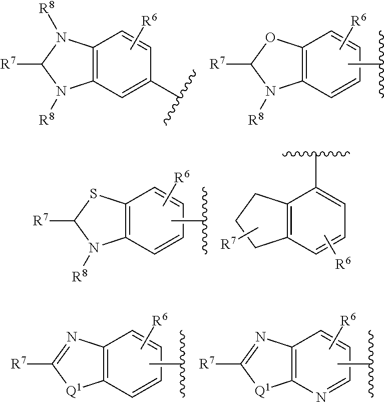 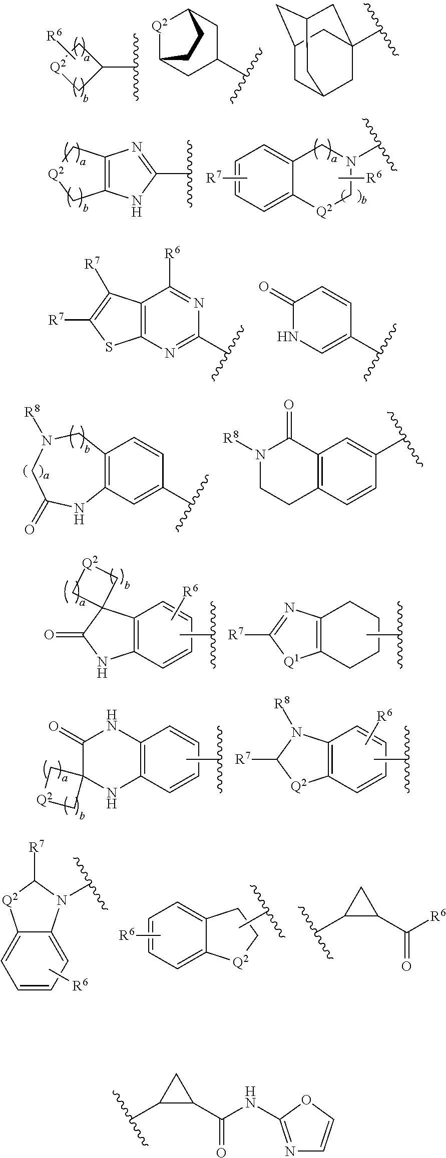
wherein R⁶ and R⁷ are independently selected from: —H, -D, —OH, C₁-C₄ alkyl, alkoxy, thioalkoxy, C₃-C₆ cycloalkyl or halogenated derivatives thereof, halogen, —(CH₂)ₐNR′R″, —NHR⁸, —C(═O)OR′, —C(═O)R⁹, —C(═NH)R⁹, —NO₂, —CN, -Ph, —SO₂—N R′R″, ═O, ═NR′, —SO₂—C₁-C₄ alkyl, or
R⁶ and R⁷ are independently selected from the following substructures: 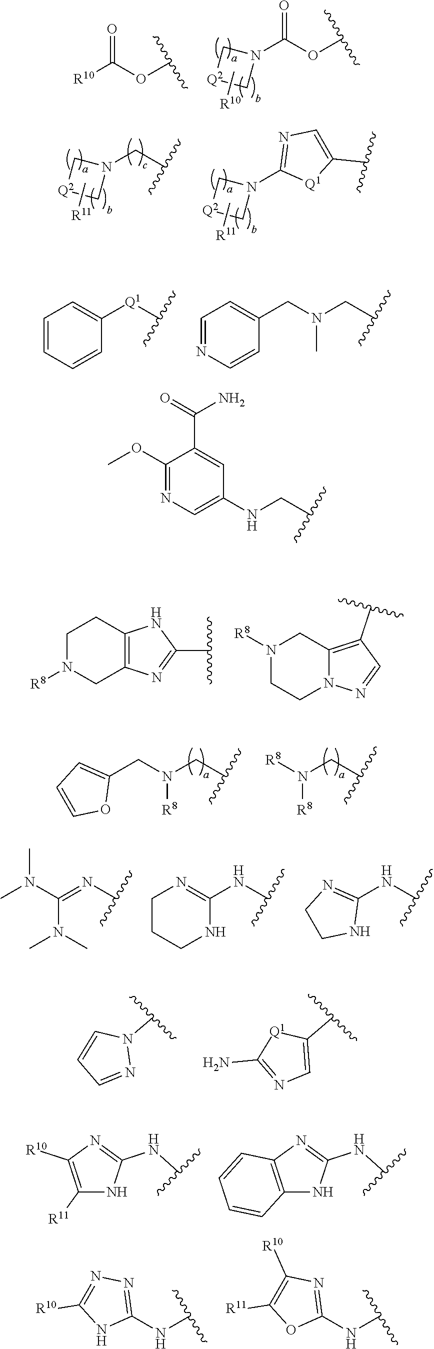 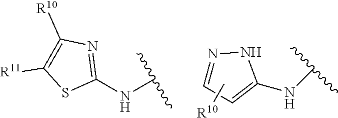
R⁸ is —H, -D, —OH, C₁-C₆ alkyl, C₃-C₆ cycloalkyl or halogenated derivatives thereof, —(CH₂)ₐNR′R″, —C(═O)OR′, —C(═O)R⁹, —C(═NH)R⁹, —(CH₂)ₐPh, —(CH₂)ₐPy, —SO₂—C₁-C₄ alkyl or R^{a} is selected from the following substructures: 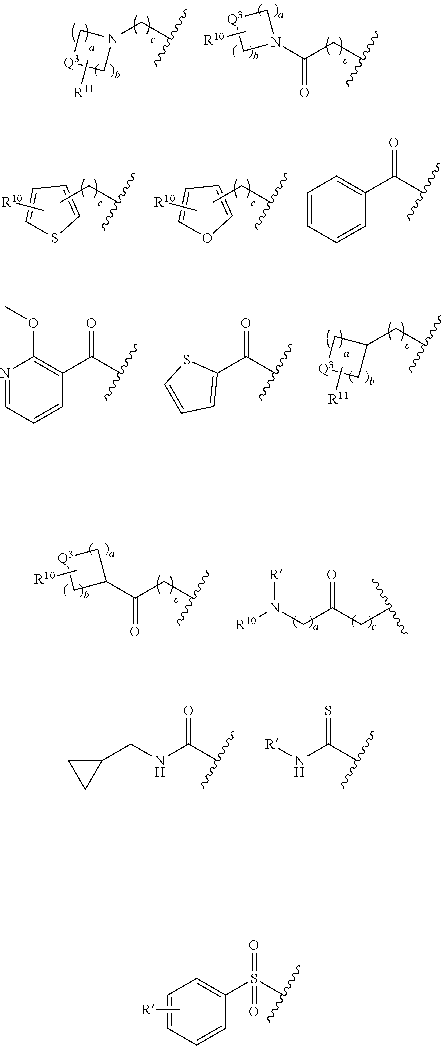
R⁹ is —NR′R″, C₁-C₄ alkyl, or halogenated derivatives thereof or is selected from the following substructures: 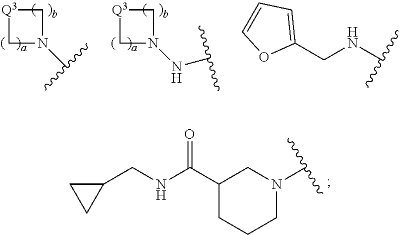
R¹⁰ and R¹¹ are independently selected from —H, C₁-C₄ alkyl, C₃-C₆ cycloalkyl or halogenated derivatives thereof, —OR′, —C(═O)OR′, —C(═O)R′, and halogen;
Q¹ is CH₂, O, S, or NR⁸;
Q² and Q³ are independently CR′R″, CF₂, O, S, or NR⁸;
R′ and R″ are independently —H, C₁-C₄ alkyl, C₃-C₆ cycloalkyl or halogenated derivatives thereof;
a, b, and c are independently 0, 1, 2, or 3, and a and b cannot be 0 at the same time;
and R⁸ is as defined above.

16. The compound according to claim 11, wherein R¹ is selected from the following substructures: 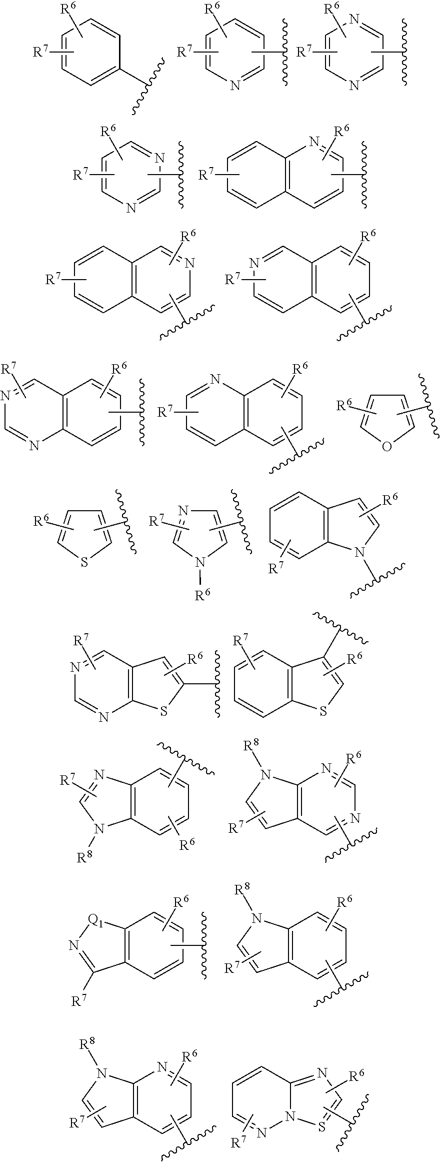 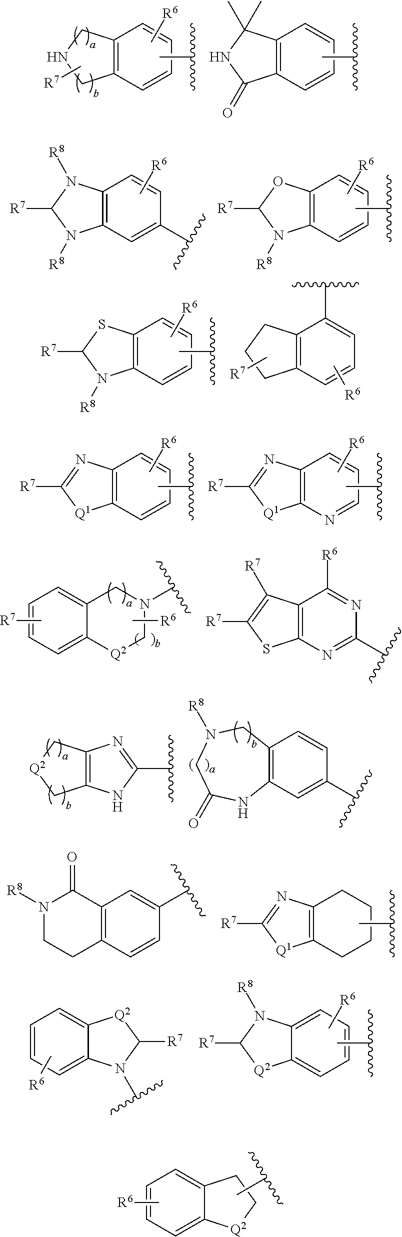
wherein R⁶ and R⁷ are independently selected from: —H, -D, —OH, C₁-C₄ alkyl, alkoxy or thioalkoxy, C₃-C₆ cycloalkyl or halogenated derivatives thereof, halogen, —(CH₂)ₐNR′R″, —NHR⁸, —C(═O)R⁹, —NO₂, -Ph, —SO₂—NR′R″, ═O, ═NR⁸, —SO₂—C₁-C₄ alkyl, or are independently selected from the following substructures: 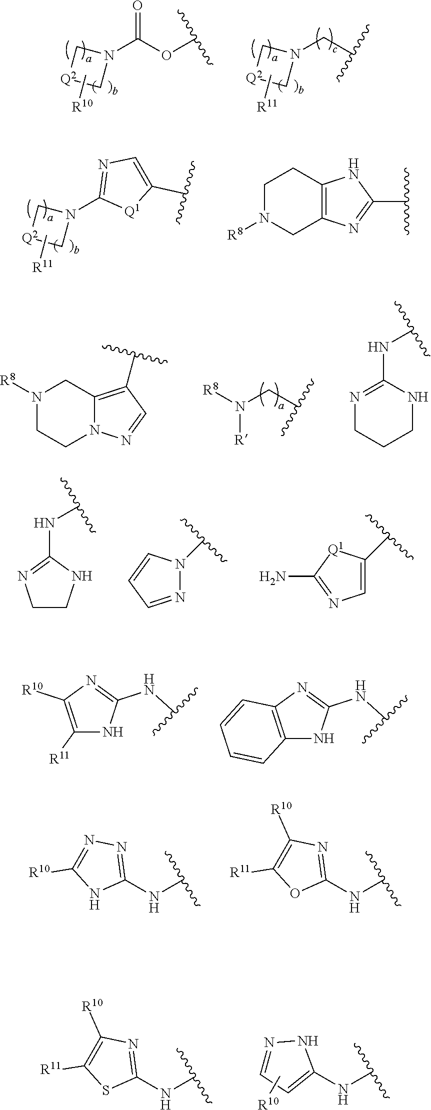
R⁸ is —H, -D, —OH, C₁-C₆ alkyl, C₃-C₆ cycloalkyl or halogenated derivatives thereof, —(CH₂)ₐNR′R″, —C(═O)OR′, —C(═O)R⁹, —C(═NH)R⁹, —SO₂—C₁-C₄ alkyl or R⁸ is selected from the following substructures: 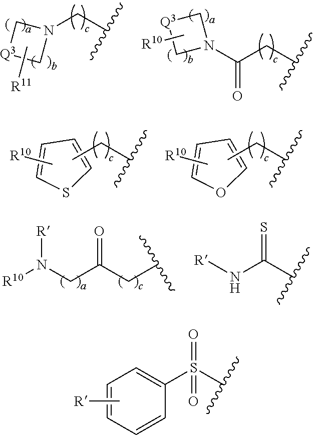
R⁹ is —NR′R″, C₁-C₄ alkyl, or halogenated derivatives thereof or is selected from the following substructures: 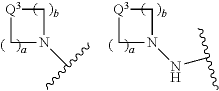
R¹⁰ and R¹¹ are independently selected from —H, C₁-C₄ alkyl, C₃-C₆ cycloalkyl or halogenated derivatives thereof, —OR′, —C(═O)OR′, —C(═O)R′, and halogen;
Q¹ is CH₂, O, S, or NR⁸;
Q² and Q³ are independently CR′R″, CF₂, O, S, or NR⁸;
R′ and R″ are independently —H, C₁-C₄ alkyl, C₃-C₆ cycloalkyl or halogenated derivatives thereof;
a, b, and c are independently 0, 1, 2, or 3, and a and b cannot be 0 at the same time;
and R⁸ is as defined above.

17. The compound according to claim 11, wherein R¹ is selected from the following substructures: 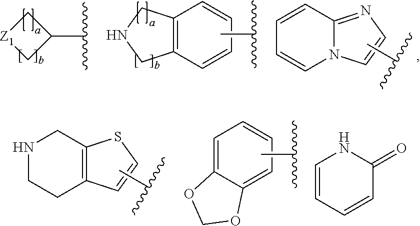
wherein a, b are independently 0, 1, 2, or 3, and a and b cannot be 0 at the same time;
Z₁ is CH₂, NH, or O;
and wherein at least one H of R¹ is optionally substituted with: halogen, —(CH₂)ₙ-Q4-Q5-Rd;
n=0, 1 or 2;
Q4 is absent, —SO₂—, —NH—, —N(C₁-C₅ alkyl)-, —NHC(═O)—, —N(C₁-C₅ alkyl)C(═O)— or —C(═O)—;
Q5 is absent, C₁-C₅ alkylene, —NH—, —(C₁-C₅ alkylene)-NH—C(═O)— or —N(C₁-C₈alkyl);
Rd is —OH, C₁-C₅ alkyl, C₁-C₅ haloalkyl, —NR′R″, C₁-C₅ alkoxy, 5 or 6 membered heteroaryl including 1 to 3 N, 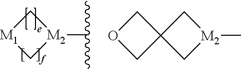
wherein e and f are independently 1 or 2;
M1 is CH₂, O, NH or SO₂, M₂ is CH or N and wherein at least one H of Rd is optionally substituted with OH, halogen, C₁-C₅alkyl, C₁-C₅ haloalkyl, —C(═O)—(C₁-C₅alkyl), —C(═O)O(C₁-C₅alkyl); —NH—C(═O)—O(C₁-C₅alkyl), —NR′R″, 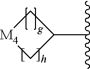
wherein g and h are independently 0, or 1, but cannot be 0 at the same time;
M4 is CH₂, O, NH and at least one H of M4 is optionally substituted with halogen, C₁-C₅ alkyl, C₃-C₆ cycloalkyl or —C(═O)—O(C₁-C₅alkyl);
R′ and R″ are independently —H or C₁-C₄ alkyl.

18. The compound according to claim 11, which is selected from:
N′-(2,2-difluoroacetyl)-4-((4-(4-((4,5-dihydro-1H-imidazol-2-yl)amino)phenyl)-1H-1,2,3-triazol-1-yl)methyl)benzohydrazide,
4-((4-(2-aminobenzo[d]thiazol-6-yl)-1H-1,2,3-triazol-1-yl)methyl)-N′-(2,2-difluoroacetyl)-3-fluorobenzohydrazide,
N′-(2,2-difluoroacetyl)-6-((4-(p-tolyl)-1H-1,2,3-triazol-1-yl)methyl)nicotinohydrazide,
N′-(2,2-difluoroacetyl)-6-((4-(m-tolyl)-1H-1,2,3-triazol-1-yl)methyl)nicotinohydrazide,
N-(3-(1-(4-(2-(2,2-difluoroacetyl)hydrazine-1-carbonyl)-2-fluorobenzyl)-1H-1,2,3-triazol-4-yl)phenyl)acetamide,
N′-(2,2-difluoroacetyl)-6-((4-phenyl-1H-1,2,3-triazol-1-yl)methyl)nicotinohydrazide,
N′-(2,2-difluoroacetyl)-6-((4-(2-fluorophenyl)-1H-1,2,3-triazol-1-yl)methyl)nicotinohydrazide,
N′-(2,2-difluoroacetyl)-5-fluoro-6-((4-(pyridin-2-yl)-1H-1,2,3-triazol-1-yl)methyl)nicotinohydrazide,
N′-(2,2-difluoroacetyl)-6-((5-phenyl-2H-tetrazol-2-yl)methyl)nicotinohydrazide,
N′-(2,2-difluoroacetyl)-6-((5-(thiophen-2-yl)-2H-tetrazol-2-yl)methyl)nicotinohydrazide,
N′-(2,2-difluoroacetyl)-6-((4-(3-fluorophenyl)-1H-1,2,3-triazol-1-yl)methyl)nicotinohydrazide,
N′-(2,2-difluoroacetyl)-6-((4-(thiophen-2-yl)-1H-1,2,3-triazol-1-yl)methyl)nicotinohydrazide,
N′-(2,2-difluoroacetyl)-6-((4-(pyridin-2-yl)-1H-1,2,3-triazol-1-yl)methyl)nicotinohydrazide,
6-((4-(1H-pyrrolo[2,3-b]pyridin-5-yl)-1H-1,2,3-triazol-1-yl)methyl)-N′-(2,2-difluoroacetyl)nicotinohydrazide,
4-(1-(4-(2-aminobenzo[d]thiazol-6-yl)-1H-1,2,3-triazol-1-yl)ethyl)-N′-(2,2-difluoroacetyl)-3-fluorobenzohydrazide,
4-((4-(2-aminobenzo[d]thiazol-6-yl)-1H-1,2,3-triazol-1-yl)methyl)-2-chloro-N′-(2,2-difluoroacetyl)benzohydrazide,
4-((4-(2-aminobenzo[d]thiazol-6-yl)-1H-1,2,3-triazol-1-yl)methyl)-N′-(2,2-difluoroacetyl)benzohydrazide,
6-((4-(1H-indazol-4-yl)-1H-1,2,3-triazol-1-yl)methyl)-N′-(2,2-difluoroacetyl)nicotinohydrazide,
4-((4-(1H-indol-4-yl)-1H-1,2,3-triazol-1-yl)methyl)-N′-(2,2-difluoroacetyl)-3-fluorobenzohydrazide,
4-((4-(2-aminobenzo[d]thiazol-6-yl)-1H-1,2,3-triazol-1-yl)methyl)-3-chloro-N′-(2,2-difluoroacetyl)benzohydrazide,
6-((4-(2-aminobenzo[d]thiazol-6-yl)-1H-1,2,3-triazol-1-yl)methyl)-N′-(2,2-difluoroacetyl)nicotinohydrazide,
6-(1-(4-(6-aminopyridin-3-yl)-1H-1,2,3-triazol-1-yl)ethyl)-N′-(2,2-difluoroacetyl)nicotinohydrazide,
6-((4-(1H-indazol-4-yl)-1H-1,2,3-triazol-1-yl)methyl)-N′-(2,2-difluoroacetyl)-5-fluoronicotinohydrazide,
4-((4-(6-aminopyridin-3-yl)-1H-1,2,3-triazol-1-yl)methyl)-3,5-difluoro-N′-(2,2,2-trifluoroacetyl)benzohydrazide,
4-((4-(6-aminopyridin-3-yl)-1H-1,2,3-triazol-1-yl)methyl)-3-fluoro-N′-(2,2,2-trifluoroacetyl)benzohydrazide,
4-((4-(6-aminopyridin-3-yl)-1H-1,2,3-triazol-1-yl)methyl)-2-fluoro-N′-(2,2,2-trifluoroacetyl)benzohydrazide,
4-((4-(6-aminopyridin-3-yl)-1H-1,2,3-triazol-1-yl)methyl)-N′-(2,2-difluoroacetyl)benzohydrazide,
4-((4-(6-aminopyridin-3-yl)-1H-1,2,3-triazol-1-yl)methyl)-N′-(2,2-difluoroacetyl)-3,5-difluorobenzohydrazide,
4-((4-(6-aminopyridin-3-yl)-1H-1,2,3-triazol-1-yl)methyl)-N′-(2,2-difluoroacetyl)-3-fluorobenzohydrazide,
5-[[4-(2-amino-1,3-benzothiazol-6-yl)triazol-1-yl]methyl]-N′-(2,2-difluoroacetyl)thiophene-2-carbohydrazide, and
5-[[4-(6-aminopyridin-3-yl)triazol-1-yl]methyl]-N′-(2,2-difluoroacetyl)thiophene-2-carbohydrazide.

19. A method of treating one or more HDAC6-mediated diseases selected from the group consisting of chemotherapy-related cognitive impairment (CRCI), graft rejection, GVHD, myositis, diseases associated with abnormal lymphocyte functions, multiple myeloma, non-Hodgkin lymphoma, peripheral neuropathies, autoimmune diseases, inflammatory diseases, cancer and neurodegenerative diseases, ocular diseases in a subject in need thereof, comprising administration of an effective amount of the compound of claim 11, alone or in combination with one or more pharmaceutically acceptable excipients.
